SEQUENCE LISTING (34 sequences)

SEQ ID NO: 1 - DNA, 3531 bp, Bacillus thuringiensis
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcctgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctcttttggcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtgcgctggtacaatacgggattagagcgtgtatggggaccggattctagagattgggtaaggtataatcaatttagaagagagctaacacttactgtattagatatcgttgctctattctcaaattatgatagtcgaaggtatccaattcgaacagtttcccaattaacaagagaaatttatacgaacccagtattagaaaattttgatggtagttttcgtggaatggctcagagaatagaacagaatattaggcaaccacatcttatggatatccttaatagtataaccatttatactgatgtgcatagaggctttaattattggtcagggcatcaaataacagcttctcctgtagggttttcaggaccagaattcgcattccctttatttgggaatgcggggaatgcagctccacccgtacttgtctcattaactggtttggggatttttagaacattatcttcacctttatatagaagaattatacttggttcaggcccaaataatcaggaactgtttgtccttgatggaacggagttttcttttgcctccctaacgaccaacttgccttccactatatatagacaaaggggtacagtcgattcactagatgtaataccgccacaggataatagtgtaccacctcgtgcgggatttagccatcgattgagtcatgttacaatgctgagccaagcagctggagcagtttacaccttgagagctccaacgttttcttggcagcatcgcagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctactacaaatttacaattccatacatcaattgacggaagacctattaatcagggtaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaaggcggtgaatgagctgtttacttcttccaatcaaatcgggttaaaaacagatgtgacggattatcatattgatcaagtatccaatttagttgagtgtttatcagatgaattttgtctggatgaaaaacaagaattgtccgagaaagtcaaacatgcgaagcgacttagtgatgagcggaatttacttcaagatccaaacttcagagggatcaatagacaactagaccgtggctggagaggaagtacggatattaccatccaaggaggcgatgacgtattcaaagagaattacgttacgctattgggtacctttgatgagtgctatccaacgtatttatatcaaaaaatagatgagtcgaaattaaaagcctatacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttcttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagagaatccttgtgaatttaacagagggtatagggattacacgccactaccagttggttatgtgacaaaagaattagaatacttcccagaaaccgataaggtatggattgagattggagaaacggaaggaacatttatcgtggacagcgtggaattactccttatggaggaatag

SEQ ID NO: 2 - protein, 1176 aa, Bacillus thuringiensis
MDNNPNINECIPYNCLSNPEVEVLGGERIETGYTPIDISLSLTQFLLSEFVPGAGFVLGLVDIIWGIFGPSQWDAFPVQIEQLINQRIEEFARNQAISRLEGLSNLYQIYAESFREWEADPTNPALREEMRIQFNDMNSALTTAIPLLAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFSNYDSRRYPIRTVSQLTREIYTNPVLENFDGSFRGMAQRIEQNIRQPHLMDILNSITIYTDVHRGFNYWSGHQITASPVGFSGPEFAFPLFGNAGNAAPPVLVSLTGLGIFRTLSSPLYRRIILGSGPNNQELFVLDGTEFSFASLTTNLPSTIYRQRGTVDSLDVIPPQDNSVPPRAGFSHRLSHVTMLSQAAGAVYTLRAPTFSWQHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKQELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVLPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 3 - DNA, 3468 bp, Bacillus thuringiensis
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagatcatgctgtacgctggtacaatacgggattagagcgtgtatggggaccggattctagagattggataagatataatcaatttagaagagaattaacactaactgtattagatatcgtttctctatttccgaactatgatagtagaacgtatccaattcgaacagtttcccaattaacaagagaaatttatacaaacccagtattagaaaattttgatggtagttttcgaggctcggctcagggcatagaaggaagtattaggagtccacatttgatggatatacttaacagtataaccatctatacggatgctcatagaggagaatattattggtcagggcatcaaataatggcttctcctgtagggttttcggggccagaattcacttttccgctatatggaactatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctcctatgttctcttggatacatcgtagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctaccacaaatttacaattccatacatcaattgacggaagacctattaatcaggggaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaaggcggtgaatgagctgtttacttcttccaatcaaatcgggttaaaaacagatgtgacggattatcatattgatcaagtatccaatttagttgagtgtttatctgatgaattttgtctggatgaaaaaaaagaattgtccgagaaagtcaaacatgcgaagcgacttagtgatgagcggaatttacttcaagatccaaactttagagggatcaatagacaactagaccgtggctggagaggaagtacggatattaccatccaaggaggcgatgacgtattcaaagagaattacgttacgctattgggtacctttgatgagtgctatccaacgtatttatatcaaaaaatagatgagtcgaaattaaaagcctatacccgttaccaattaagagggtatatcgaagatagtcaagacttagaaatctatttaattcgctacaatgccaaacacgaaacagtaaatgtgccaggtacgggttccttatggccgctttcagccccaagtccaatcggaaaatgtgcccatcattcccatcatttctccttggacattgatgttggatgtacagacttaaatgaggacttaggtgtatgggtgatattcaagattaagacgcaagatggccatgcaagactaggaaatctagaatttctcgaagagaaaccattagtaggagaagcactagctcgtgtgaaaagagcggagaaaaaatggagagacaaacgtgaaaaattggaatgggaaacaaatattgtttataaagaggcaaaagaatctgtagatgctttatttgtaaactctcaatatgatagattacaagcggataccaacatcgcgatgattcatgcggcagataaacgcgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaccgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgtgtagaagaggaagtatatccaaacaacacggtaacgtgtaatgattatactgcgactcaagaagaatatgagggtacgtacacttctcgtaatcgaggatatgacggagcctatgaaagcaattcttctgtaccagctgattatgcatcagcctatgaagaaaaagcatatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattacttcttatggaggaataa

SEQ ID NO: 4 - protein, 1155 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 5 - DNA, 3900 bp, Bacillus thuringiensis
cttaaaaaaattcaagaaatatgatgttgattcttagagcaatgaaagagttattaccctgtttaaggtgtcatgattctcctagaaaagtaggcagatgaagaatggacgatatgcatacggataacgtgactcgtaaaaagtacgcttcgtatatgatatgtatatttcaacgtacagtcatcaaaatgtaacatgggaggcgtgaaaatattgtgatgaataattggcatataccgtttactccccacatagaatgtgcagagcggttttagaggtacttgaaacccataaatgaaagtagaagggggtgaataccatgcatgaatcgcaagaaggtagagatgttccgaatgggataactaaacataaacaccatataccttttcagtgtatcgtttcccttccatcaggttttcaaattgaaaagccgaatgatttgaaacttgtttacgatgtaagtcatttgtctatgacgaaagatacgtgtaaaaaacgtattgagattgatgaatgtggacaagtagaaattgacttacaagtattaaagattaagggagtcctttcttttatcgggaatttttctattgaacccattgtgtgtgaaaacatgtatacaacggtggacagaaatccatctatttccttaagttttcaagatacggtatatgtggaccatattttaaaatatagtgtgcagcaattaccacattatgtcattgatggtgatcatattcaagtacgtgagttacaaatcaaattgatgaaagaaaatccccaatctgctcaaatatctggtcttttttgttttgtgtatgagtaagaaccgaaggtttataaaaaatttaaatatacactatagtgtctttatcaggattgaaagtgaaaaactataggtatagatgttagctagttatcttaataattcaatagtatccaaaggatttaactatgcacgataaaaatagttaataggaggaaaagattttatgctaaaatatcattttcctaatgtatgtgaagatgaattaattaatatatattcttatggggattttaaaggacaaggaaaatatatatgcctctttaaaattgaaaaccaatcattcttattttggagaaacgataaaggaaataaaatatatacaaatttagaatctatcagtgtagaaataataaacacgaataatacgtataatcaaagtcagaatgtatgcccacaagatttagttgatacgtataatcaaagtcagaatgtatgcccacaagatttagttgatacgtataatcaaagtcagaatgtatgcccacaagatttagttgatacgtataaccaaagtcagaatgtatgcccacaagatttagttgatacgtataatcaaagtcagaatgtatgcccacaagatttagttgatacgtataaccaaagtcagaatgtatgcccacaagatttagttgatacgtataaccaaagtcagaatgtatacacacaagatttaattgatacgtataaccaaagtcagaatgtatgcccacaagatttagttgatacgtataaccaaagtcagaatgtatgcccacaagatttagttgatacgtataaccaaagtcagaatgtatgcccacaagatttgaatgtatacacacaagatttaattgatacgtataatcaaagtcagaattgtgattgtggttgtaagtagtaagtagtaagtagtttcttaaacatactcgttattatcaaaagagtttagttttaatataaaactagatatttaaggaggaattttatatgaataatgtattgaatagtggaagaacaactatttgtgatgcgtataatgtagtagcccatgatccatttagttttgaacataaatcattagataccatccaaaaagaatggatggagtggaaaagaacagatcatagtttatatgtagctcctgtagtcggaactgtgtctagttttttgctaaagaaagtggggagtcttattggaaaaaggatattgagtgaattatgggggataatatttcctagtggtagtacaaatctaatgcaagatattttaagggagacagaacaattcctaaatcaaagacttaatacagatacccttgctcgtgtaaatgcagaattgatagggctccaagcgaatataagggagtttaatcaacaagtagataattttttaaaccctactcaaaaccctgttcctttatcaataacttcttcggttaatacaatgcagcaattatttctaaatagattaccccagttccagatacaaggataccagttgttattattacctttatttgcacaggcagccaatatgcatctttcttttattagagatgttattcttaatgcagatgaatggggtatttcagcagcaacattacgtacgtatcgagattacctgagaaattatacaagagattattctaattattgtataaatacgtatcaaactgcgtttagagggttaaacacccgtttacacgatatgttagaatttagaacatatatgtttttaaatgtatttgaatatgtatccatttggtcattgtttaaatatcagagtcttatggtatcttctggcgctaatttatatgctagcggtagtggaccacagcagacacaatcatttacagcacaaaactggccatttttatattctcttttccaagttaattcgaattatatattatctggtattagtggtactaggctttctattaccttccctaatattggtggtttaccgggtagtactacaactcattcattgaatagtgccagggttaattatagcggaggagtttcatctggtctcataggggcgactaatctcaatcacaactttaattgcagcacggtcctccctcctttatcaacaccatttgttagaagttggctggattcaggtacagatcgagagggcgttgctacctctacgaattggcagacagaatcctttcaaacaactttaagtttaaggtgtggtgctttttcagcccgtggaaattcaaactatttcccagattattttatccgtaatatttctggggttcctttagttattagaaacgaagatctaacaagaccgttacactataaccaaataagaaatatagaaagtccttcgggaacacctggtggagcacgggcctatttggtatctgtgcataacagaaaaaataatatctatgccgctaatgaaaatggtactatgatccatttggcgccagaagattatacaggatttactatatcgccaatacatgccactcaagtgaataatcaaactcgaacatttatttctgaaaaatttggaaatcaaggtgattccttaagatttgaacaaagcaacacgacagctcgttatacgcttagagggaatggaaatagttacaatctttatttaagagtatcttcaataggaaattcaactattcgagttactataaacggtagagtttatactgtttcaaatgttaataccactacaaataacgatggagttaatgataatggagctcgtttttcagatattaatatcggtaatatagtagcaagtgataatactaatgtaacgctagatataaatgtgacattaaactccggtactccatttgatctcatgaatattatgtttgtgccaactaatcttccaccactttattaaggtttgagtgaatgtacaattagtattttattctatcataaatttaatagaaaattcttaaacatattgacggaactaaatgatatataattatggatattagagggtgtcttaaagtagtaaaattcttactctgagacaccctctttatttttttatatccaaatcggatgaaatatgggagaaatcatttcaagttaacctaa

SEQ ID NO: 6 - protein, 633 aa, Bacillus thuringiensis
MNNVLNSGRTTICDAYNVVAHDPFSFEHKSLDTIQKEWMEWKRTDHSLYVAPVVGTVSSFLLKKVGSLIGKRILSELWGIIFPSGSTNLMQDILRETEQFLNQRLNTDTLARVNAELIGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSLMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANENGTMIHLAPEDYTGFTISPIHATQVNNQTRTFISEKFGNQGDSLRFEQSNTTARYTLRGNGNSYNLYLRVSSIGNSTIRVTINGRVYTVSNVNTTTNNDGVNDNGARFSDINIGNIVASDNTNVTLDINVTLNSGTPFDLMNIMFVPTNLPPLY

SEQ ID NO: 7 - DNA, 1935 bp, Bacillus thuringiensis
atgaatccgaacaatcgaagtgaacatgatacaataaaaactactgaaaataatgaggtgccaactaaccatgttcaatatcctttagcggaaactccaaatccaacactagaagatttaaattataaagagtttttaagaatgactgcagataataatacggaagcactagatagctctacaacaaaagatgtcattcaaaaaggcatttccgtagtaggtgatctcctaggcgtagtaggtttcccgtttggtggagcgcttgtttcgttttatacaaactttttaaatactagttggccaagtgaagacccgtggaaggcttttatggaacaagtagaagcattgatggatcagaaaatagctgattatgcaaaaaataaagctcttgcagagttacagggccttcaaaataatgtcgaagattatgtgagtgcattgagttcatggcaaaaaaatcctgtgagttcacgaaatccacatagccaggggcggataagagagctgttttctcaagcagaaagttattttcgtaattcaatgccttcgtttgcaatttctggatacgaggttctatttctaacaacatatgcacaagctgccaacacacatttatttttactaaaagacgctcaaatttatggagaagaatggggatacgaaaaagaagatattgctgaattttataaaagacaactaaaacttacgcaagaatatactgaccattgtgtcaaatggtataatgttgggttagataaattaagaggttcatcttatgaatcttgggtaaactttaaccgttatcgcagagagatgacattaacagtattagatttaattgcactatttccattgtatgatgttcggctatacccaaaagaagttaaaaccgaattaacaagagacgttttaacagatccaattgtcggagtcaacaaccttaggggctatggaacaaccttctctaatatagaaaattatattcgaaaaccacatctatttaactatctgcgtagaattcaatttcacacgcggttccaaccaggatattatggaaatgactctttcaattattggtccggtaattatgtttcaactagaccaagcataggatcaaatgatataatcacatctccattctatggaaataaatccagtgaacctgtacaaaatttagaatttaatggagaaaaagtctatagagccgtagcaaatacaaatcttgcggtctggccgtccgctgtatattcaggtgttacaaaagtggaatttagccaatataatgatcaaacagatgaagcaagtacacaaacgtacgactcaaaaagaaatgttggcgcggtcagctgggattctatcgatcaattgcctccagaaacaacagatgaacctctagaaaagggatatagccatcaactcaattatgtaatgtgctttttaatgcagggtagtagaggaacaatcccagtgttaacttggacacataaaagtgtagacttttttaacatgattgattcgaaaaaaattacacaacttccgttagtaaaggcatataagttacaatctggtgcttccgttgtcgcaggtcctaggtttacaggaggagatatcattcaatgcacagaaaatggaagtgcggcaactatttacgttacaccggatgtgtcgtactctcaaaaatatcgagctagaattcattatgcttctacatctcagataacatttacactcagtttagacggggcaccatttaatcaatactatttcgataaaacgataaataaaggagacacattaacgtataattcatttaatttagcaagtttcagcacaccattcgaattatcagggaataacttacaaataggcgtcacaggattaagtgctggagataaagtttatatagacaaaattgaatttattccagtgaattaa

SEQ ID NO: 8 - protein, 644 aa, Bacillus thuringiensis
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTSWPSEDPWKAFMEQVEALMDQKIADYAKNKALAELQGLQNNVEDYVSALSSWQKNPVSSRNPHSQGRIRELFSQAESYFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFNYLRRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 9 - DNA, 3543 bp, Bacillus thuringiensis
atgaatccttatcaaaataaaaatgaatatgaaacattaaatgcttcacaaaaaaaattaaatatatctaataattatacaagatatccaatagaaaatagtccaaaacaattattacaaagtacaaattataaagattggctcaatatgtgtcaacagaatcagcagtatggtggagattttgaaacttttattgatagtggtgaactcagtgcctatactattgtagttgggaccgtactgactggtttcgggttcacaacacccttaggacttgctttaataggttttggtacattaataccagttctttttccagcccaagaccaatctaacacatggagtgactttataacacaaactaaaaatattataaaaaaagaaatagcatcaacatatataagtaatgctaataaaattttaaacaggtcgtttaatgttatcagcacttatcataatcaccttaaaacatgggagaataatccaaacccacaaaatactcaggatgtaaggacacaaatccagctagttcattaccattttcaaaatgtcattccagagcttgtaaactcttgtcctcctaatcctagtgattgcgattactataacatactagtattatctagttatgcacaagcagcaaacttacatctgactgtattaaatcaagccgtcaaatttgaagcgtatttaaaaaacaatcgacaattcgattatttagagcctttgccaacagcaattgattattatccagtattgactaaagctatagaagattacactaattattgtgtaacaacttataaaaaaggattaaatttaattaaaacgacgcctgatagtaatcttgatggaaatataaactggaacacatacaatacgtatcgaacaaaaatgactactgctgtattagatgttgttgcactctttcctaattatgatgtaggtaaatatccaataggtgtccaatctgaacttactcgagaaatttatcaggtacttaacttcgaagaaagcccctataaatattatgactttcaatatcaagaggattcacttacacgtagaccgcatttatttacttggcttgattctttgaatttttatgaaaaagcgcaaactactcctaataattttttcaccagccattataatatgtttcattacacacttgataatatatcccaaaaatctagtgtttttggaaatcacaatgtaactgataaattaaaatctcttggtttggcaacaaatatttatatttttttattaaatgtcataagcttagataataaatatctaaatgattataataatattagtaaaatggatttttttataactaatggtactagacttttggagaaagaacttacagcaggatctgggcaaataacttatgatgtaaataaaaatattttcgggttaccaattcttaaacgaagagagaatcaaggaaaccctaccctttttccaacatatgataactatagtcatattttatcatttattaaaagtcttagtatccctgcaacatataaaactcaagtgtatacgtttgcttggacacactctagtgttgatcctaaaaatacaatttatacacatttaactacccaaattccagctgtaaaagcgaattcacttgggactgcttctaaggttgttcaaggacctggtcatacaggaggggatttaattgatttcaaagatcatttcaaaattacatgtcaacactcaaattttcaacaatcgtattttataagaattcgttatgcttcaaatggaagcgcaaatactcgagctgttataaatcttagtatcccaggggtagcagaactgggtatggcactcaaccccactttttctggtacagattatacgaatttaaaatataaagattttcagtacttagaattttctaacgaggtgaaatttgctccaaatcaaaacatatctcttgtgtttaatcgttcggatgtatatacaaacacaacagtacttattgataaaattgaatttctgccaattactcgttctataagagaggatagagagaaacaaaaattagaaacagtacaacaaataattaatacattttatgcaaatcctataaaaaacactttacaatcagaacttacagattatgacatagatcaagccgcaaatcttgtggaatgtatttctgaagaattatatccaaaagaaaaaatgctgttattagatgaagttaaaaatgcgaaacaacttagtcaatctcgaaatgtacttcaaaacggggattttgaatcggctacgcttggttggacaacaagtgataatatcacaattcaagaagatgatcctatttttaaagggcattaccttcatatgtctggggcgagagacattgatggtacgatatttccgacctatatattccaaaaaattgatgaatcaaaattaaaaccgtatacacgttacctagtaaggggatttgtaggaagtagtaaagatgtagaactagtggtttcacgctatggggaagaaattgatgccatcatgaatgttccagctgatttaaactatctgtatccttctacctttgattgtgaagggtctaatcgttgtgagacgtccgctgtgccggctaacattgggaacacttctgatatgttgtattcatgccaatatgatacagggaaaaagcatgtcgtatgtcaggattcccatcaatttagtttcactattgatacaggggcattagatacaaatgaaaatataggggtttgggtcatgtttaaaatatcttctccagatggatacgcatcattagataatttagaagtaattgaagaagggccaatagatggggaagcactgtcacgcgtgaaacacatggagaagaaatggaacgatcaaatggaagcaaaacgttcggaaacacaacaagcatatgatgtagcgaaacaagccattgatgctttattcacaaatgtacaagatgaggctttacagtttgatacgacactcgctcaaattcagtacgctgagtatttggtacaatcgattccatatgtgtacaatgattggttgtcagatgttccaggtatgaattatgatatctatgtagagttggatgcacgagtggcacaagcgcgttatttgtatgatataagaaatattattaaaaatggtgattttacacaaggggtaatggggtggcatgtaactggaaatgcagacgtacaacaaatagatggtgtttctgtattggttctatctaattggagtgctggcgtatctcaaaatgtccatctccaacataatcatgggtatgtcttaggtgttattgccaaaaaagaaggacctggaaatgggtatgtcacgcttatggattgggaggagaatcaagaaaaattgacgtttacgtcttgtgaagaaggatatattacgaagacagtagatgtattcccagatacagatcgtgtacgaattgagataggcgaaaccgaaggttcgttttatatcgaaagcattgaattaatttgcatgaacgagtga

SEQ ID NO: 10 - protein, 1180 aa, Bacillus thuringiensis
MNPYQNKNEYETLNASQKKLNISNNYTRYPIENSPKQLLQSTNYKDWLNMCQQNQQYGGDFETFIDSGELSAYTIVVGTVLTGFGFTTPLGLALIGFGTLIPVLFPAQDQSNTWSDFITQTKNIIKKEIASTYISNANKILNRSFNVISTYHNHLKTWENNPNPQNTQDVRTQIQLVHYHFQNVIPELVNSCPPNPSDCDYYNILVLSSYAQAANLHLTVLNQAVKFEAYLKNNRQFDYLEPLPTAIDYYPVLTKAIEDYTNYCVTTYKKGLNLIKTTPDSNLDGNINWNTYNTYRTKMTTAVLDVVALFPNYDVGKYPIGVQSELTREIYQVLNFEESPYKYYDFQYQEDSLTRRPHLFTWLDSLNFYEKAQTTPNNFFTSHYNMFHYTLDNISQKSSVFGNHNVTDKLKSLGLATNIYIFLLNVISLDNKYLNDYNNISKMDFFITNGTRLLEKELTAGSGQITYDVNKNIFGLPILKRRENQGNPTLFPTYDNYSHILSFIKSLSIPATYKTQVYTFAWTHSSVDPKNTIYTHLTTQIPAVKANSLGTASKVVQGPGHTGGDLIDFKDHFKITCQHSNFQQSYFIRIRYASNGSANTRAVINLSIPGVAELGMALNPTFSGTDYTNLKYKDFQYLEFSNEVKFAPNQNISLVFNRSDVYTNTTVLIDKIEFLPITRSIREDREKQKLETVQQIINTFYANPIKNTLQSELTDYDIDQAANLVECISEELYPKEKMLLLDEVKNAKQLSQSRNVLQNGDFESATLGWTTSDNITIQEDDPIFKGHYLHMSGARDIDGTIFPTYIFQKIDESKLKPYTRYLVRGFVGSSKDVELVVSRYGEEIDAIMNVPADLNYLYPSTFDCEGSNRCETSAVPANIGNTSDMLYSCQYDTGKKHVVCQDSHQFSFTIDTGALDTNENIGVWVMFKISSPDGYASLDNLEVIEEGPIDGEALSRVKHMEKKWNDQMEAKRSETQQAYDVAKQAIDALFTNVQDEALQFDTTLAQIQYAEYLVQSIPYVYNDWLSDVPGMNYDIYVELDARVAQARYLYDIRNIIKNGDFTQGVMGWHVTGNADVQQIDGVSVLVLSNWSAGVSQNVHLQHNHGYVLGVIAKKEGPGNGYVTLMDWEENQEKLTFTSCEEGYITKTVDVFPDTDRVRIEIGETEGSFYIESIELICMNE

SEQ ID NO: 11 - DNA, 3408 bp, Bacillus thuringiensis
atgaattcaggctatccgttagcgaatgacttacaagggtcaatgaaaaacacgaactataaagattggctagccatgtgtgaaaataaccaacagtatggcgttaatccagctgcgattaattcttcttcagttagtaccgctttaaaagatgctggagctatccttaaatttgtaaacccacctgcaggatctgtcttaaccgtacttagcgcggtgcttcctattctttggccgactaatactccaacgcctgaaagagtttggaatgatttcatgaccaatacagggaatcttattgatcaaactgtaacagcttatgtacgaacagatgcaaatgcaaaaatgacggttgtgaaagattatttagatcaatatacaactaaatttaacacttggaaaagagagcctaataaccagtcctatagaacagcagtaataactcaatttaacttaaccagtgccaaacttcgagagaccgcagtttattttagcaacttagtaggttatgaattattgttattaccaatatacgcacaagtagcaaatttcaatttacttttaataagagatggccctcataaatgcacaagaatggtctatgcacgatcgtgtgaccaactatataacactatggtgcagtacactaaagaatatattgcacatagcattacatggtataataaaggtttagatgtacttagaaataaatctaatggacaatggattacgtttaatgattataaaagagagatgactattcaagtattagatatactcgctctttttgccagttatgatccacgtcgataccctgcggacaaaatagataatacgaaactatcaaaaacagaatttacaagagagatttatacagctttagtagaatctccttctagtaaatctatagcagcactggaggcagcacttacacgagatgttcatttattcacttggctaaagagagtagatttctggaccaatactatatatcaagatttaagatttttatctgccaataaaattgggttttcatatacaaattcttctgcaatgcaagaaagtggaatttatggaagttctggtttggttcaaatctatctcatcaaattcaacttaattctaattgttataaaacttctatcacagatactagctccccctctaatcgagttacaaaaatggatttctacaaaatttgatggtactcttgcctcttataattcaaatataacaccaactcctgaaggtttaaggaccacattttttggattttcaacaaatgagaacacacctaatcaaccaactgtaaatgattatacgcatattttaagctatataaaaactgatgttatagattataacagtaacagggtttcatttgcttggacacataacattgttgaccctaataatcaaatatacacagatgctatcacacaagttccggccgtaaaatctaacttcttgaatgcaacagctagagtaatcaagggacctggtcatacagggggggatctagttgctcttacaagcaatggtactctatcgggaggcagaatggagattcaatgtaaaacaagtatttttaatgatcctacaagaagttacggattacgcatacgttatgctgcaaatagtccaattgtgattgaatgtgatcatatgtattacaaggagtttctagaggaacaacgattagtacagaactacgtttcaagacctaataatataatacctacagatttaaaatatgaagagtttagatacaaagatcctaatgatgcaattgtaccgatgagattatcttctaatcaactgataactatagctattcaaccattaaacatgacttcaaataatcaagtgattattgacagaatcgaaattattccaatcactcaatctgtattagatgagacagagaaccaaaatttagaatcagaacgagaagttgtgaatgcactgtttacaaatgacgcgaaagatgcattaaacattggaacgacagattatgacatagatcaagccgcaaatcttgtggaatgtatttctgaaggaattatatccaaagaaaaaatgctcttattagatgaagttaaaaatgcgaaacaacttagtcaatctcgaaatgtacttcaaaacggggattttgaatcgcgtacgcttggttggacaacaagtgataatatcacaattcaagaagatgatcctatttttaaagggcattaccttcatatgtctggggcgagagacattgatggtacgatatttccgacctatatattccaaaaaattgatgaatcaaaattaaaaccgtatacacgttacctagtaaggggatttgtaggaagtagtaaagatgtagaactagtggtttcacgctatggggaagaaattgatgccatcatgaatgttccagctgatttaaactatctgtatccttctacctttgattgtgaagggctaatcgttgtgagcgtccgctgtgccgctaacatttgggacacttctgatatgttgtattcatgccaatatgatacagggaaaaagcatgtcgtatgtcaggattcccatcaatttagtttcactattgatacaggggcattagatacaaatgaaaatataggggtttgggtcatgtttaaaatatcttctccagatggatacgcatcattagataatttagaagtaattgaaagagggccaatagatggggaagcactgtcacgcgtgaaacacatggagaagaaatggaacgatcaaatggaagcaaaacgttcggaaacacaacaagcatatgatgtagcgaaacaagccattgatgctttattcacaaatgtacaagatgaggctttacagtttgatacgacactcgctcaaattcagtacgctgagtatttggtacaatcgattccatatgtgtacaatgattggttgtcagatgttccaggtatgaattatgatatctatgtagagttggatgcacgagtggcacaagcgcgttatttgtatgatacaagaaatattattaaaaatgttgattttacacaaggggtaatggggtggcatgtaactggaaatgcagacgtacaacaaatagatggtgtttctgtattggttctatctaattggagtgctggcgtatctcaaaatgtccatctccaacataatcatgggtatgtcttacgtgttattgccaaaaaagaaggacctggaaatgggtatgtcacgcttatggattgtgaggagaatcaagaaaaattgacgtttacgtcttgtgaagaaggatatattacgaagacagtagatgtattcccagatacagatcgtgtacgaattgagataggcgaaaccgaaggttcgttttatatcgaaagcattgaattaatttgcatgaacgagtga

SEQ ID NO: 12 - protein, 1135 aa, Bacillus thuringiensis
MNSGYPLANDLQGSMKNTNYKDWLAMCENNQQYGVNPAAINSSSVSTALKDAGAILKFVNPPAGSVLTVLSAVLPILWPTNTPTPERVWNDFMTNTGNLIDQTVTAYVRTDANAKMTVVKDYLDQYTTKFNTWKREPNNQSYRTAVITQFNLTSAKLRETAVYFSNLVGYELLLLPIYAQVANFNLLLIRDGPHKCTRMVYARSCDQLYNTMVQYTKEYIAHSITWYNKGLDVLRNKSNGQWITFNDYKREMTIQVLDILALFASYDPRRYPADKIDNTKLSKTEFTREIYTALVESPSSKSIAALEAALTRDVHLFTWLKRVDFWTNTIYQDLRFLSANKIGFSYTNSSAMQESGIYGSSGLVQIYLIKFNLILIVIKLLSQILAPPLIELQKWISTKFDGTLASYNSNITPTPEGLRTTFFGFSTNENTPNQPTVNDYTHILSYIKTDVIDYNSNRVSFAWTHNIVDPNNQIYTDAITQVPAVKSNFLNATARVIKGPGHTGGDLVALTSNGTLSGGRMEIQCKTSIFNDPTRSYGLRIRYAANSPIVIECDHMYYKEFLEEQRLVQNYVSRPNNIIPTDLKYEEFRYKDPNDAIVPMRLSSNQLITIAIQPLNMTSNNQVIIDRIEIIPITQSVLDETENQNLESEREVVNALFTNDAKDALNIGTTDYDIDQAANLVECISEGIISKEKMLLLDEVKNAKQLSQSRNVLQNGDFESRTLGWTTSDNITIQEDDPIFKGHYLHMSGARDIDGTIFPTYIFQKIDESKLKPYTRYLVRGFVGSSKDVELVVSRYGEEIDAIMNVPADLNYLYPSTFDCEGLIVVSVRCAANIWDTSDMLYSCQYDTGKKHVVCQDSHQFSFTIDTGALDTNENIGVWVMFKISSPDGYASLDNLEVIERGPIDGEALSRVKHMEKKWNDQMEAKRSETQQAYDVAKQAIDALFTNVQDEALQFDTTLAQIQYAEYLVQSIPYVYNDWLSDVPGMNYDIYVELDARVAQARYLYDTRNIIKNVDFTQGVMGWHVTGNADVQQIDGVSVLVLSNWSAGVSQNVHLQHNHGYVLRVIAKKEGPGNGYVTLMDCEENQEKLTFTSCEEGYITKTVDVFPDTDRVRIEIGETEGSFYIESIELICMNE

SEQ ID NO: 13 - DNA, 1932 bp, Bacillus thuringiensis
atggaagatagttctttagatactttaagtatagttaatgaaacagactttccattatataataattataccgaacctactattgcgccagcattaatagcagtagctcccatcgcacaatatcttgcaacagctatagggaaatgggcggcaaaggcagcattttcaaaagtactatcacttatattcccaggttctcaacctgctactatggaaaaagttcgtacagaagtggaaacacttataaatcaaaaattaagccaagatcgagtcaatatattaaacgcagaatatagggggattattgaggttagtgatgtatttgatgcgtatattaaacaaccaggttttacccctgcaacagccaagggttattttctaaatctaagtggtgctataatacaacgattacctcaatttgaggttcaaacatatgaaggagtatctatagcactttttactcaaatgtgtacacttcatttaactttattaaaagacggaatcctagcagggagtgcatggggatttactcaagctgatgtagattcatttataaaattatttaatcaaaaagtattagattacaggaccagattaatgagaatgtacacagaagagttcggaagattgtgtaaagtcagtcttaaagatggattgacgttccggaatatgtgtaatttatatgtgtttccatttgctgaagcctggtctttaatgagatatgaaggattaaaattacaaagctctctatcattatgggattatgttggtgtctcaattcctgtaaattataatgaatggggaggactagtttataagttattaatgggggaagttaatcaaagattaacaactgttaaatttaattattctttcactaatgaaccagctgatataccagcaagagaaaatattcgtggcgtccatcctatatacgatcctagttctgggcttacaggatggataggaaacggaagaacaaacaattttaattttgctgataacaatggcaatgaaattatggaagttagaacacaaactttttatcaaaatccaaataatgagcctatagcgcctagagatattataaatcaaattttaactgcgccagcaccagcagacctattttttaaaaatgcagatataaatgtaaagttcacacagtggtttcagtctactctatatgggtggaacattaaactcggtacacaaacggttttaagtagtagaaccggaacaataccaccaaattatttagcatatgatggatattatattcgtgctatttcagcttgcccaagaggagtctcacttgcatataatcacgatcttacaacactaacatataatagaatagagtatgattcacctactacagaaaatattattgtagggtttgcaccagataatactaaggacttttattctaaaaaatctcactatttaagtgaaacgaatgatagttatgtaattcctgctctgcaatttgctgaagtttcagatagatcatttttagaagatacgccagatcaagcaacagacggcagtattaaatttgcacgtactttcattagtaatgaagctaagtactctattagactaaacaccgggtttaatacggcaactagatataaattaattatcagggtaagagtaccttatcgcttacctgctggaatacgggtacaatctcagaattcgggaaataatagaatgctaggcagttttactgcaaatgctaatccagaatgggtggattttgtcacagatgcatttacatttaacgatttagggattacaacttcaagtacaaatgctttatttagtatttcttcagatagtttaaattctggagaagagtggtatttatcgcagttgtttttagtaaaagaatcggcctttacgacgcaaattaatccgttactaaagtag

SEQ ID NO: 14 - protein, 643 aa, Bacillus thuringiensis
MEDSSLDTLSIVNETDFPLYNNYTEPTIAPALIAVAPIAQYLATAIGKWAAKAAFSKVLSLIFPGSQPATMEKVRTEVETLINQKLSQDRVNILNAEYRGIIEVSDVFDAYIKQPGFTPATAKGYFLNLSGAIIQRLPQFEVQTYEGVSIALFTQMCTLHLTLLKDGILAGSAWGFTQADVDSFIKLFNQKVLDYRTRLMRMYTEEFGRLCKVSLKDGLTFRNMCNLYVFPFAEAWSLMRYEGLKLQSSLSLWDYVGVSIPVNYNEWGGLVYKLLMGEVNQRLTTVKFNYSFTNEPADIPARENIRGVHPIYDPSSGLTGWIGNGRTNNFNFADNNGNEIMEVRTQTFYQNPNNEPIAPRDIINQILTAPAPADLFFKNADINVKFTQWFQSTLYGWNIKLGTQTVLSSRTGTIPPNYLAYDGYYIRAISACPRGVSLAYNHDLTTLTYNRIEYDSPTTENIIVGFAPDNTKDFYSKKSHYLSETNDSYVIPALQFAEVSDRSFLEDTPDQATDGSIKFARTFISNEAKYSIRLNTGFNTATRYKLIIRVRVPYRLPAGIRVQSQNSGNNRMLGSFTANANPEWVDFVTDAFTFNDLGITTSSTNALFSISSDSLNSGEEWYLSQLFLVKESAFTTQINPLLK

SEQ ID NO: 15 - DNA, 2175 bp, Bacillus thuringiensis
atgcaaaataacaactttaataccacagaaattaataatatgattaatttccctatgtataatggtagattagaaccttctctagctccagcattaatagcagtagctccaattgctaaatatttagcaacagctcttgctaaatgggctgtaaaacaagggtttgcaaaattaaaatccgagatattccccggtaatacgcctgctactatggataaggttcgtattgaggtacaaacacttttagaccaaagattacaagatgacagagttaagattttagaaggtgaatacaaaggaattattgacgtgagtaaagtttttactgattatgttaatcaatctaaatttgagactggaacagctaataggcttttttttgatacaagtaaccaattaataagcagattgcctcaatttgagattgcaggatatgaaggagtatccatttcactttttactcagatgtgtacatttcatttgggtttattaaaagatggaattttagcaggaagcgattggggatttgctcctgcagataaagacgctcttatttgccaattcaatagatttgtcaatgaatataatactcgactgatggtattgtactcaaaagaatttggacggttattagcaaaaaatcttaatgaagccttgaactttagaaatatgtgtagtttatatgtctttcctttttctgaagcatggtctttattaaggtatgaaggaacaaaattagaaaacacgctttcattatggaattttgtgggtgaaagtatcaataatatatctcctaatgattggaaaggtgcgctttataaattgttaatgggagcacctaatcaaagattaaacaatgttaagtttaattatagttatttttctgatactcaagcgacaatacatcgtgaaaacattcatggtgtcctgccaacatataatggaggaccaacaattacaggatggatagggaatgggcgtttcagcggacttagttttccttgtagtaatgaattagaaattacaaaaataaaacaggaaataacttacaatgataaagggggaaatttcaattcaatagttcctgctgctacgcgcaatgaaattctaactgctaccgttccaacatcagctgatccattttttaaaaccgctgatattaactggaaatatttctctccgggtctttactctggatggaatattaaatttgatgatacagtcactttaaaaagtagagtaccaagtattataccttcaaatatattaaagtatgatgattattatattcgtgccgtttcagcctgtccaaaaggcgtatcacttgcatataaccatgattttttaacgttaacatataataaattagaatatgatgcacctactacacaaaatatcattgtaggattttcaccagataatactaagagtttttataggagcaactctcattatctaagtacaacagatgatgcctatgtaattcctgctttacaattttctacagtctcagatagatcattcttagaagatacaccagatcaagcaacagatggcagtattaaatttacggatactgttcttgggaatgaggcaaaatattctattagactaaatactggatttaatacagctactaggtatagattaattatacgttttaaagcgcctgctcgtttggctgctggtatacgtgtacgttctcaaaattcagggaataataagttattaggtggtattcctgtagagggtaattctggatggatagattatattacagattcatttacttttgatgaccttgggattacaacttcaagtacaaatgctttctttagtattgattcagatggtgtaaatgcttctcaacaatggtatttgtctaaattaattttagtaaaagaatccagttttacgactcagattccattaaaaccatacgttattgtacgttgtccggatactttttttgtgagcaacaattcaagtagtacgtacgaacaaggctataacaacaattacaaccagaattctagcagtatgtacgatcaaggctataacaatagctataatccaaactctggttgtacgtgtaatcaagactataacaatagctataaccaaaactctggctgtacatgtaaccaagggtataacaataactatcctaaataa

SEQ ID NO: 16 - protein, 724 aa, Bacillus thuringiensis
MQNNNFNTTEINNMINFPMYNGRLEPSLAPALIAVAPIAKYLATALAKWAVKQGFAKLKSEIFPGNTPATMDKVRIEVQTLLDQRLQDDRVKILEGEYKGIIDVSKVFTDYVNQSKFETGTANRLFFDTSNQLISRLPQFEIAGYEGVSISLFTQMCTFHLGLLKDGILAGSDWGFAPADKDALICQFNRFVNEYNTRLMVLYSKEFGRLLAKNLNEALNFRNMCSLYVFPFSEAWSLLRYEGTKLENTLSLWNFVGESINNISPNDWKGALYKLLMGAPNQRLNNVKFNYSYFSDTQATIHRENIHGVLPTYNGGPTITGWIGNGRFSGLSFPCSNELEITKIKQEITYNDKGGNFNSIVPAATRNEILTATVPTSADPFFKTADINWKYFSPGLYSGWNIKFDDTVTLKSRVPSIIPSNILKYDDYYIRAVSACPKGVSLAYNHDFLTLTYNKLEYDAPTTQNIIVGFSPDNTKSFYRSNSHYLSTTDDAYVIPALQFSTVSDRSFLEDTPDQATDGSIKFTDTVLGNEAKYSIRLNTGFNTATRYRLIIRFKAPARLAAGIRVRSQNSGNNKLLGGIPVEGNSGWIDYITDSFTFDDLGITTSSTNAFFSIDSDGVNASQQWYLSKLILVKESSFTTQIPLKPYVIVRCPDTFFVSNNSSSTYEQGYNNNYNQNSSSMYDQGYNNSYNPNSGCTCNQDYNNSYNQNSGCTCNQGYNNNYPK

SEQ ID NO: 17 - DNA, 1947 bp, Bacillus thuringiensis
atgcattattatgggaataggaatgaatatgacatattaaatgcttcatcaaacgattcaaacatgtctaatacttatccgaggtatccgttagcaaatccacaacaagatttaatgcaaaatacaaattataaagattggcttaatgtatgtgaagggtatcatatagaaaatcctagagaagcaagcgttagagctggacttggtaagggattaggtatagttagtacaatcgtagggttctttggtggttctattatcttagatacaattggattgttttaccaaatttcagagctactttggccagaggatgatacccagcaatacacttggcaagatattatgaatcatgtagaagatcttatagacaaacgaataactgaggttatacgaggaaatgcaattagaacattagcagatttacagggtaaagttgatgattataacaattggttgaagaaatggaaagacgatccaaaatctacaggtaatttaagcaccttagtaaccaagtttacggctcttgattcagattttaatggtgctataaggacagttaataatcaggggagtccaggttatgagttacttttattgcctgtctatgcacaaattgcgaatctgcatttacttttattacgggatgctcagatttatggagataaatggtggagcgcacgagctaatgctcgtgataattattaccaaatacaattagagaaaacaaaggaatatacagaatattgtataaattggtataataagggtttaaatgattttagaacagcaggtcaatgggtaaactttaatcgttatcgtagagaaatgactcttactgtattagatattatttcaatgttccctatttatgacgcgagattatatcctacagaagtaaaaaccgaactaactagggaaatttattcagatgttattaatggggagatatatggacttatgactccttatttttcttttgagaaagctgaatcactttatacaagggcaccccatctcttcacttggctaaaaggatttcgatttgtaaccaattctatttcttattggacatttttatcaggtggtcaaaataagtattcttatactaataattctagtattaacgagggctcttttaggggacaggacacagattatggtgggacttcttctaccattaatattccatcaaattcgtatgtatataatttatggacggaaaattatgaatatatttatccttggggtgatcctgtaaatattacaaaaatgaatttttctgtaacagataataattcttcaaaagaattaatttatggtgcacacagaacgaataaacctgttgttcggacagattttgattttctcactaataaagagggaactgagttagcaaaatataatgattataatcatattttatcctatatgttaattaatggggaaacgtttggtcagaaacgtcatggttattcgtttgcttttacacatagtagtgttgatcctaataataccattgcagcgaataaaattacgcaaattcctgtagtgaaagcttcgagtataaatggatcgatttcaattgaaaaaggtcccggatttacgggaggagatttggtaaagatgagagcagattcaggtttaactatgcgttttaaagctgaattattagataaaaaatatcgtgttcgaatacgttataaatgtaactacagttctaaattaatactacgaaaatggaaaggggaaggttatatacaacaacaaattcacaatatttctcccacatatggagccttttcttatttagagtcttttactataactacgacagaaaatatatttgatttgacaatggaggtaacatatccgtatggtagacagtttgttgaagatataccatctcttatattagataaaatcgaattcctcccaactaactga

SEQ ID NO: 18 - protein, 648 aa, Bacillus thuringiensis
MHYYGNRNEYDILNASSNDSNMSNTYPRYPLANPQQDLMQNTNYKDWLNVCEGYHIENPREASVRAGLGKGLGIVSTIVGFFGGSIILDTIGLFYQISELLWPEDDTQQYTWQDIMNHVEDLIDKRITEVIRGNAIRTLADLQGKVDDYNNWLKKWKDDPKSTGNLSTLVTKFTALDSDFNGAIRTVNNQGSPGYELLLLPVYAQIANLHLLLLRDAQIYGDKWWSARANARDNYYQIQLEKTKEYTEYCINWYNKGLNDFRTAGQWVNFNRYRREMTLTVLDIISMFPIYDARLYPTEVKTELTREIYSDVINGEIYGLMTPYFSFEKAESLYTRAPHLFTWLKGFRFVTNSISYWTFLSGGQNKYSYTNNSSINEGSFRGQDTDYGGTSSTINIPSNSYVYNLWTENYEYIYPWGDPVNITKMNFSVTDNNSSKELIYGAHRTNKPVVRTDFDFLTNKEGTELAKYNDYNHILSYMLINGETFGQKRHGYSFAFTHSSVDPNNTIAANKITQIPVVKASSINGSISIEKGPGFTGGDLVKMRADSGLTMRFKAELLDKKYRVRIRYKCNYSSKLILRKWKGEGYIQQQIHNISPTYGAFSYLESFTITTTENIFDLTMEVTYPYGRQFVEDIPSLILDKIEFLPTN

SEQ ID NO: 19 - DNA, 717 bp, Artificial Sequence
  Synthetic construct modified green fluorescent protein
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcacctatggtgttcaatgcttttcaagatacccagatcatatgaagcggcacgacttcttcaagagcgccatgcctgagggatacgtgcaggagaggaccatcttcttcaaagacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 20 - DNA, 711 bp, Artificial Sequence
  fluorescent protein derived from Discosoma sp.
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 21 - DNA, 567 bp, Ricinus communis
atgatcttcccgaagcaataccctattatcaactttacgactgctggagcaacagtgcagtcttatacaaactttattcgagcggttagaggtcgtttaacagtacttcctaaccgtgtaggtttaccgattaatcaacgttttatccttgtggaactatcaaatcacgcagagttaagcgtaacattggcactagacgttactaatgcttatgtagtgggatatagagcaggtaacagcgcgtatttcttccatcctgataatcaagaagacgctgaagctattactcacctatttactgacgtgcaaaatcgatatacatttgcttttggaggaaattacgatcgcttggaacaacttgcaggtaacttacgtgaaaacatcgaactagggaatgggccacttgaagaagcgatcagtgcgttatactattatagtactggtggaactcaattaccgactctagctagaagctttatcatctgtatacaaatgattagtgaagcggctcgttttcagtatatagaaggcgaaatgcgtacaagaattcgctataatcgccgtagt

SEQ ID NO: 22 - DNA, 981 bp, Yersinia pestis
atgattagagcctacgaacaaaacccacaacattttattgaggatctagaaaaagttagggtggaacaacttactggtcatggttcttcagttttagaagaattggttcagttagtcaaagataaaaatatagatatttccattaaatatgatcccagaaaagattcggaggtttttgccaatagagtaattactgatgatatcgaattgctcaagaaaatcctagcttattttctacccgaggatgccattcttaaaggcggtcattatgacaaccaactgcaaaatggcatcaagcgagtaaaagagttccttgaatcatcgccgaatacacaatgggaattgcgggcgttcatggcagtaatgcatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtagtggcaccataaatatccatgataaatccattaatctcatggataaaaatttatatggttatacagatgaagagatttttaaagccagcgcagagtacaaaattctcgagaaaatgcctcaaaccaccattcaggtggatgggagcgagaaaaaaatagtctcgataaaggactttcttggaagtgagaataaaagaaccggggcgttgggtaatctgaaaaactcatactcttataataaagataataatgaattatctcactttgccaccacctgctcggataagtccaggccgctcaacgacttggttagccaaaaaacaactcagctgtctgatattacatcacgttttaattcagctattgaagcactgaaccgtttcattcagaaatatgattcagtgatgcaacgtctgctagatgacacgtctggtaaatga

SEQ ID NO: 23 - DNA, 288 bp, Mycobacterium tuberculosis
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcatag

SEQ ID NO: 24 - DNA, 1017 bp, Mycobacterium tuberculosis
atgcagcttgtcgacagggttcgtggcgccgtcacgggtatgtcgcgtcgactcgtggtcggggccgtcggcgcggccctagtgtcgggtctggtcggcgccgtcggtggcacggcgaccgcgggggcattttcccggccgggcttgccggtggagtacctgcaggtgccgtcgccgtcgatgggccgtgacatcaaggtccaattccaaagtggtggtgccaactcgcccgccctgtacctgctcgacggcctgcgcgcgcaggacgacttcagcggctgggacatcaacaccccggcgttcgagtggtacgaccagtcgggcctgtcggtggtcatgccggtgggtggccagtcaagcttctactccgactggtaccagcccgcctgcggcaaggccggttgccagacttacaagtgggagaccttcctgaccagcgagctgccggggtggctgcaggccaacaggcacgtcaagcccaccggaagcgccgtcgtcggtctttcgatggctgcttcttcggcgctgacgctggcgatctatcacccccagcagttcgtctacgcgggagcgatgtcgggcctgttggacccctcccaggcgatgggtcccaccctgatcggcctggcgatgggtgacgctggcggctacaaggcctccgacatgtggggcccgaaggaggacccggcgtggcagcgcaacgacccgctgttgaacgtcgggaagctgatcgccaacaacacccgcgtctgggtgtactgcggcaacggcaagccgtcggatctgggtggcaacaacctgccggccaagttcctcgagggcttcgtgcggaccagcaacatcaagttccaagacgcctacaacgccggtggcggccacaacggcgtgttcgacttcccggacagcggtacgcacagctgggagtactggggcgcgcagctcaacgctatgaagcccgacctgcaacgggcactgggtgccacgcccaacaccgggcccgcgccccagggcgcctag

SEQ ID NO: 25 - DNA, 978 bp, Mycobacterium tuberculosis
atgacagacgtgagccgaaagattcgagcttggggacgccgattgatgatcggcacggcagcggctgtagtccttccgggcctggtggggcttgccggcggagcggcaaccgcgggcgcgttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatcccaaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggctga

SEQ ID NO: 26 - DNA, 1023 bp, Mycobacterium tuberculosis
atgacgttcttcgaacaggtgcgaaggttgcggagcgcagcgacaaccctgccgcgccggctggctatcgcggctatgggggctgtcctggtttacggtctggtcggtaccttcggcgggccggccaccgcgggcgcattctctaggcccggtcttccagtggaatatctgcaggtgccatccgcgtcgatgggccgcgacatcaaggtccagttccagggcggcggaccgcacgcggtctacctgctcgacggtctgcgggcccaggatgactacaacggctgggacatcaacaccccggccttcgaggagtactaccagtcagggttgtcggtgatcatgcccgtgggcggccaatccagtttctacaccgactggtatcagccctcgcagagcaacggccagaactacacctacaagtgggagaccttccttaccagagagatgcccgcctggctacaggccaacaagagcgtgtccccgacaggcaacgcggcggtgggtctttcgatgtcgggcggttccgcgctgatcctggccgcgtactacccgcagcagttcccgtacgccgcgtcgttgtcgggcttcctcaacccgtccgagggctggtggccgacgctgatcggcctggcgatgaacgactcgggcggttacaacgccaacagcatgtggggtccgtccagcgacccggcctggaagcgcaacgacccaatggttcagattccccgcctggtcgccaacaacacccggatctgggtgtactgcggtaacggcacacccagcgacctcggcggcgacaacataccggcgaagttcctggaaggcctcaccctgcgcaccaaccagaccttccgggacacctacgcggccgacggtggacgcaacggggtgtttaacttcccgcccaacggaacacactcgtggccctactggaacgagcagctggtcgccatgaaggccgatatccagcatgtgctcaacggcgcgacacccccggccgcccctgctgcgccggccgcctga

SEQ ID NO: 27 - DNA, 1536 bp, Mycobacterium tuberculosis
cggcttcggaataggcattgcccccgatgtgcgggcgccgctcgaggacgagcacgcgcttgtcgagttgggtggccacgcgctcggcaatcgtcaggccgaagaatcctgagccgacgacgaaaaggtcaaaacgagcggtcatcggttgcatagggtaaccgaccttgctggcaaaacccgatttggcagctcgtggcggtcatggcccgaacgggtttcaccgcaggtgcgcatggccgaccagtgtggttggccggaggtcgtttggtcgcgattgcctcacgattcgatataaccactctagtcacatcaaccacactcgtaccatcgagcgtgtgggttcatgccatgcactcgcgaccgcgggagccggcgaacccggcgccacacataatccagattgaggagacttccgtgccgaaccgacgccgacgcaagctctcgacagccatgagcgcggtcgccgccctggcagttgcaagtccttgtgcatattttcttgtctacgaatcaaccgaaacgaccgagcggcccgagcaccatgaattcaagcaggcggcggtgttgaccgacctgcccggcgagctgatgtccgcgctatcgcaggggttgtcccagttcgggatcaacataccgccggtgcccagcctgaccgggagcggcgatgccagcacgggtctaaccggtcctggcctgactagtccgggattgaccagcccgggattgaccagcccgggcctcaccgaccctgcccttaccagtccgggcctgacgccaaccctgcccggatcactcgccgcgcccggcaccaccctggcgccaacgcccggcgtgggggccaatccggcgctcaccaaccccgcgctgaccagcccgaccggggcgacgccgggattgaccagcccgacgggtttggatcccgcgctgggcggcgccaacgaaatcccgattacgacgccggtcggattggatcccggggctgacggcacctatccgatcctcggtgatccaacactggggaccataccgagcagccccgccaccacctccaccggcggcggcggtctcgtcaacgacgtgatgcaggtggccaacgagttgggcgccagtcaggctatcgacctgctaaaaggtgtgctaatgccgtcgatcatgcaggccgtccagaatggcggcgcggtcgcgccggcagccagcccgccggtcccgcccatccccgcggccgcggcggtgccaccgacggacccaatcaccgtgccggtcgcctaagccccgggtcggccgaaaacgcacccgcggccaaggcgtcggtcattgcttcggcccgtcacaattactcgcctaagggtcgctaggtgttctcgagagttttatcgcaccgattccgtgtcgtctcattaataccaatagaaacacacgtaacatcagctggtgccgtcccgcacccgcgcgccgacgacgctgctcaccgcgatggcagcgaccgtcgtcatcgtcgcgtggatagcgaatcgtccacccgccagctcccat

SEQ ID NO: 28 - DNA, 1419 bp, Mycobacterium tuberculosis
atgagacggaatcgccgtggctcgccagcgcgaccggccgcacggtttgtccgtccggcaattccgtcggctttgagtgtggccctgctggtatgcacaccggggctggctaccgccgatccacagacggacaccatcgccgcgctgattgccgacgtcgccaaggccaaccagcgcctgcaagacctgagcgacgaggttcaggccgaacaggaaagcgttaacaaggcgatggtcgacgtggaaaccgctcgggacaacgctgccgcggccgaagacgacctggaggtcagccagcgcgcggttaaggacgccaacgcggcgatcgccgcggctcagcaccggttcgacaccttcgcggcggccacctacatgaacggtccctcggtcagctacctcagcgcgagcagccccgacgagatcattgccactgtgaccgccgccaagacccttagcgccagttcccaagcggtgatggccaacctgcagcgggcccggaccgagcgggtgaacacggagtcggcggcgcggctagccaagcagaaggctgataaggccgccgccgacgcaaaggccagccaggatgccgcggtggcggcgctcaccgagacccggcggaagttcgatgaacagcgcgaggaggtccaacgcctggccgccgagcgcgatgcggctcaagcccgactgcaggcggccaggttggttgcctggtcctcggagggtggtcagggtgcgccgccgttccggatgtgggatcccggatcgggccctgccggtgggcgtgcatgggatggcttgtgggaccccacgctgcccatgatccccagcgccaacatccccggcgacccgatcgcggtagtgaaccaggtgttggggatctcggcaacgtcagcgcaggtcaccgccaatatggggcgcaagttcctggagcagctgggcatcttgcagcccaccgataccggcatcaccaacgctccggcgggctcggcccagggccggattccgcgagtttatgggcgccaggcttctgaatacgtgatccgccgcggcatgtcacagatcggggtgccctattcctggggcggcggcaatgccgcgggcccgagcaagggcatcgactccggggccggcaccgtcggcttcgactgctcaggcctggtgttgtactcgtttgctggggtgggcatcaagctgccgcactactcgggttcgcagtacaacctgggccgcaagatcccgtcctcgcagatgcgccgcggcgacgtcatcttctacggcccgaacggtagccagcacgtgacgatctacctcggcaacggccagatgctcgaggcgcccgacgtcggtttgaaggtgcgggttgcgcccgtgcgcacggctggcatgaccccgtatgtggtccgatacatcgagtactag

SEQ ID NO: 29 - DNA, 522 bp, Infectious salmon anemia virus
atgaacacaagttcagcgaaacgcatgagttgccgcggtactatgcgcagtgtctggaagcctctgctgacattttccttgatgaacttgctgctgttgttacaggtggcttctttcctgtcgggctcaaaggttcctggggaggatggtatctcaagtacgtcaggtatgctggacctcttgaaggatcaagtgggttcattgtcaatcaacgattctatgacagagcccaaaacagagctggacccagggttgtatccgtggttgaaatggacagagacggcgtatcattcatctacgcgcagcctggcatctaccattgtgatgggtgcactgggacagcagcgagggtctggagacgggatcacaatgcgcgagctggagttgagcttagggctggacttcacttcagaatgtgattggctgaaaacatgttttgtaaacaagaattttgtgtttttgtctgagcaagaaattgctgtaaacatggaagttgaaaaatacatttgtaatgagaattaa

SEQ ID NO: 30 - DNA, 522 bp, Escherichia coli
atgaaacgttttagtctggctattctggcgctggttgttgcaaccggcgcacaagctgccagtgaaaaagtcgagatgaacctcgtcacgtcgcaaggggtagggcagtcaattggtagcgtcaccattactgaaaccgataaaggtctggagttttcgcccgatctgaaagcattaccccccggtgaacatggcttccatattcatgccaaaggaagctgccagccagccaccaaagatggcaaagccagcgccgcggaatccgcaggcgggcatcttgatccacaaaataccggtaaacatgaagggccagaaggtgccgggcatttaggcgatctgcctgcactggtcgtcaataatgacggcaaagctaccgatgccgtcatcgcgcctcgtctgaaatcactggatgaaatcaaagacaaagcgctgatggtccacgttggcggcgataatatgtccgatcaacctaaaccgctgggcggtggcggtgaacgctatgcctgtggtgtaattaagtaa

SEQ ID NO: 31 - DNA, 1653 bp, Artificial Sequence
  firefly luciferase
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 32 - DNA, 1541 bp, Bacillus thuringiensis
aagctttcagtgaagtacgtgattatacggagatgaaaattcgtacactgttaacgagaaggaaacgccgacgaaagcgtagcatcggatggcaaagatggagtaacgaatatctctacggtgtactggggctttactgagactagaaagtccttcccttgaaaagtgcagagagttttcgataaaagtgtcagccatttgataagtctcattctcataacctattgatgaagtttatagggaagctgcttgagagggaaaacctcacgaacagttcttatggggagagactggaaacaggtcacaattgatacctcgctaatcttttaaccgacaaagtttttttaaaccgtggaagtcataataacctggatattgtgaatttataaaagttaacaaatggtttatattaagacagtcataaaccaaagatttttcttctaaagctacgatagcaaaaatttcactagaaattagttatacaagcattttgtaagaattattaaaaagataaatcctgctattacgagattagtaggatgatattgtgaaaaattttttatctattcgatttaaaatatttatgaattttacataaacctcataagaaaaaatactatctatactattttaagaaatttattagaataagcggattcaaaatagccctggccataaaagtacctcagcagtagaagttttgaccaaaattaaaaaaatacccaatcaagagaatattcttaattacaatacgttttgcgaggaacatattgattgaaatttaataaatttagtcctaaaatttaaagaaatttaagtttttcatatttttatgaactaacaagaataaaaattgtgtttatttattattcttgttaaatatttgataaagagatatatttttggtcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaaatcattttatcagtttgaaaattatgtattatgataagaaagggaggaagaaaa

SEQ ID NO: 33 - DNA, 303 bp, Homo sapiens
ctggggagagtatatactgaatttagcttctgagacatgatgctcttcctttttaattaacccagaacttagcagcttatctatttctctaatctcaaaacatccttaaactgggggtgatacttgagtgagagaattttgcaggtattaaatgaactatcttcttttttttttttctttgagacagagtcttgctctgtcacccaggctggagtgcagtggcgtgatctcagctcactgcaacctccgcctcccgggttcaagtgattctcctgcctcagcctcctgagtagctgggattac

SEQ ID NO: 34 - DNA, 507 bp, Mus musculus
atggagtccgctgcagacagactggccagggcggcggcccagggccgtgtgcatgacgtgcgggcactgctggaagccggggtttcgcccaacgccccgaactctttcggtcgtaccccgattcaggtgatgatgatgggcaacgttcacgtagcagctcttctgctcaactacggtgcagattcgaactgcgaggaccccactaccttctcccgcccggtgcacgacgcagcgcgggaaggcttcctggacacgctggtggtgctgcacgggtcaggggctcggctggatgtgcgcgatgcctggggtcgcctgccgctcgacttggcccaagagcggggacatcaagacatcgtgcgatatttgcgttccgctgggtgctctttgtgttccgctgggtggtctttgtgtaccgctgggaacgtcgcccagaccgacgggcatagcttcagctcaagcacgcccagggccctggaacttcgcggccaatcccaagagcagagctaa

(19) United States
(12) Patent Application Publication
(10) Pub. No.: US 2010/0322977 A1
(43) Pub. Date: Dec. 23, 2010

(54) BIOMATERIALS, COMPOSITIONS, AND METHODS

(75) Inventors: Michael K. Chan, Hilliard, OH (US); Manoj S. Nair, Columbus, OH (US); Marianne M. Lee, Hilliard, OH (US)

Correspondence Address: STANDLEY LAW GROUP LLP, 6300 Riverside Drive, Dublin, OH, 43017, US

(73) Assignee: THE OHIO STATE UNIVERSITY RESEARCH FOUNDATION, Columbus, OH (US)

(21) Appl. No.: 12/795,485

(22) Filed: Jun. 7, 2010

Related U.S. Application Data
(60) Provisional application No. 61/184,637, filed on Jun. 5, 2009.
(60) Provisional application No. 61/313,525, filed on Mar. 12, 2010.

(51) Int. Cl. A61K 38/02; C12N 1/20; C12N 5/04; C12N 9/00; C07K 2/00; C07H 21/00; C12N 15/63; C12P 21/00; A61P 37/02

(52) U.S. Cl. 424/282.1; 435/252.1; 435/410; 435/183; 530/350; 536/23.4; 435/320.1; 514/1.1; 435/69.7

(57) ABSTRACT

Embodiments exploit the ability of the organism Bacillus thuringiensis to produce regularly shaped micrometer-sized crystals of Cry insect protoxins. In various embodiments, these crystals are used as a platform to generate various compositions that are useful for numerous applications.

### CROSS-REFERENCE TO RELATED APPLICATIONS

This non-provisional application claims the benefit of priority to U.S. Provisional Patent Application No. 61/184,637, filed Jun. 5, 2009, and U.S. Provisional Patent Application No. 61/313,525, filed Mar. 12, 2010, both of which are expressly incorporated by reference in their entirety.

### STATEMENT REGARDING FEDERALLY SPONSORED RESEARCH OR DEVELOPMENT

The present invention was made with United States Government support under Grant/Contract No. 1 R21AI081291-01 by the National Institute of Health. The United States Government may have certain rights to this invention under 35 U.S.C. §200 et seq.

### TECHNICAL FIELD

Embodiments are directed to multi-purpose biomaterials. More particularly, embodiments relate to biomaterials, compositions, and methods using Cry proteins, or crystal forming fragments thereof.

### BACKGROUND

An environmentally friendly approach to controlling pests is the use of pesticidal crystal proteins derived from the soil bacterium Bacillus thuringiensis (“Bt”), commonly referred to as “Cry proteins.” The Cry proteins are globular protein molecules which accumulate as protoxins in crystalline form during late stage of the sporulation of Bacillus thuringiensis. After ingestion by the pest, the crystals are solubilized to release protoxins in the alkaline midgut environment of the larvae. Protoxins (about 130 kDa) are converted into mature toxic fragments (about 66 kDa N-terminal region) by gut proteases. Many of these proteins are quite toxic to specific target insects, but harmless to plants and other non-targeted organisms.

Some Cry proteins have been recombinantly expressed in crop plants to provide pest-resistant transgenic plants. Among those, Bt-transgenic cotton and corn have been widely cultivated. A large number of Cry proteins have been isolated, characterized and classified based on amino acid sequence homology (Crickmore et al., 1998, Microbiol. Mol. Biol. Rev., 62: 807-813). This classification scheme provides a systematic mechanism for naming and categorizing newly discovered Cry proteins. The Cry1 classification is the best known and contains the highest number of cry genes which currently totals over 130.

To date, the use of Cry proteins has been primarily limited to pest control related applications.

### SUMMARY

Embodiments exploit the ability of the organism Bacillus thuringiensis to produce regularly shaped micrometer-sized crystals of Cry insect protoxins. In various embodiments, these crystals are used as a platform to generate various compositions that are useful for numerous applications.

Accordingly, embodiments include a cultured cell, comprising: a protein crystal formed by a plurality of a fusion polypeptide, the fusion polypeptide comprising a Cry protein, or a crystal-forming fragment thereof, fused to a heterologous polypeptide.

In exemplary embodiments, the cultured cell is a bacterium. In alternative embodiments, the cell is a eurkaryotic cell, such as a plant cell.

In some embodiments, the heterologous polypeptide is an immunogenic antigen. In other embodiments, the heterologous polypeptide is an imageable agent. In some embodiments, the imageable agent is a fluorescent protein. In alternative embodiments, the heterologous polypeptide is a blood substitute. In other embodiments, the heterologous polypeptide is a therapeutic protein and/or enzyme. In yet other embodiments, the heterologous polypeptide is an industrial enzyme.

In various embodiments, the Cry protein may be any Cry protein or a truncated crystal-forming Cry protein component, from the Bacillus thuringiensis genome. For example, the Cry protein may be Cry1Aa, Cry1Ab Cry2Aa, Cry3Aa, Cry4Aa, Cry4Ba, Cry11Aa, Cry11Ba, and Cry19Aa, their homologs, or a crystal-forming fragment thereof.

In some embodiments, the immunogenic antigen is selected from the group consisting of fbpA, fbpB, fbpC, ESAT6, erp (pirG), Rv1477, MPT53, OmpAtb, IiA, p60, MPT53, OspA.

Other embodiments include a protein crystal isolated from a bacterium, comprising: a fusion polypeptide, the fusion polypeptide comprising a Cry protein fused to a heterologous polypeptide. In some embodiments, the heterologous polypeptide is an immunogenic antigen. In other embodiments, the heterologous polypeptide is an imageable agent. In various other embodiments, the imageable agent is a fluorescent protein. In alternative embodiments, the heterologous polypeptide is a blood substitute. In yet other embodiments, the heterologous polypeptide is a therapeutic protein and/or enzyme. In other embodiments, the heterologous polypeptide is an industrial enzyme.

Other embodiments relate to a composition, comprising a Cry protein crystal chemically crosslinked to a heterologous polypeptide.

Another aspect includes a nucleic acid comprising a nucleotide sequence encoding a fusion polypeptide capable of forming crystals in vivo in a cell, the fusion polypeptide comprising a Cry protein fused to a heterologous polypeptide. Furthermore, various embodiments include an expression vector to express a fusion polypeptide.

Another embodiment relates to a bacterial endospore comprising a nucleic acid comprising a nucleotide sequence encoding a fusion polypeptide, the fusion polypeptide comprising a Cry protein fused with a heterologous polypeptide.

At least one embodiment includes a fusion polypeptide comprising a Cry protein and an immunogenic antigen.

Embodiments also include pharmaceutical compositions comprising fusion protein crystals and/or cry crystals chemically crosslinked to heterologous polypeptides, as described herein. Preferrably, the compositions additionally comprise a pharmaceutically acceptable excipient, carrier, diluent, or vehicle.

Various embodiments include a method of isolating a recombinant protein crystal from a bacterium, the method comprising: transforming the bacterium with a nucleic acid expression vector encoding a Cry protein fused to a heterologous polypeptide; growing the bacterium in culture until a spore/crystal mixture is released from the bacterium upon autolysis; centrifuging the spore/crystal mixture using a density gradient or affinity method; and isolating the purified crystals of fusion proteins.

In some embodiments, the bacterium is a Bacillus thuringiensis or a Bacillus subtilis. Accordingly, some embodiments include a method of isolating recombinant protein crystals from Bacillus thuringiensis cells, the method comprising: transforming Bacillus thuringiensis cultures with an expression vector comprising a nucleotide sequence encoding a fusion protein capable of forming a crystal in a live bacterium; the fusion protein comprising a Cry protein and a heterologous polypeptide; growing the Bacillus thuringiensis culture until a spore/crystal mixture is released from autolysed Bacillus thuringiensis cells; centrifuging the spore/crystal mixture using a density gradient; separating the crystals chromatographically; and isolating the purified crystals of fusion proteins.

Embodiments include a method of eliciting an immune response against an antigen in a subject, the method comprising administering to the subject a protein crystal comprising a Cry protein fused to a heterologous immunogenic antigen, in an amount effective to induce an immune response against the antigen in the subject. In some embodiments, the administering step is performed intranasally, orally, or intraperitoneally.

Embodiments also include a method of inducing an immune response against an antigen, the method comprising administering a protein crystal comprising a Cry protein fused to a heterologous antigen to a subject in an amount effective to induce an immune response against the antigen.

### BRIEF DESCRIPTION OF THE DRAWINGS

A better understanding of the embodiments will be obtained from a reading of the following detailed description and the accompanying drawings in which:

FIG. 1: Schematic of GFP fused Cry1Ab protein crystal in sporulating Bacillus thuringiensis bacterium

FIG. 2: Schematic of the expressed fusion protein with N-terminal GFP domain and C-terminal Cry domains.

FIG. 3: (A) Plasmid map of pHT315fusion Cry1Ab expression vector; (B) An exemplary fusion crystal expression vector in which Cry3Aa gene is placed before mCherry; (C) Plasmid map of pSB634-1Ab with GFP inserted.

FIG. 4: (A) & (B) Sequence data confirming two different regions of one exemplary GFP-Cry1Ab clone.

FIG. 5: Flowchart indicating an exemplary method for the production of the bio-crystals from Bt and its purification. The example shows a method for purifying fluorescent biocrystals using a pSB6341Ab expression plasmid.

FIG. 6: An SDS-PAGE gel of protein from Cry1Ab crystals obtained by the exemplary method shown in FIG. 5.

FIG. 7: Images of fluorescent crystals under Nikon 80i microscope: (A) Phase contrast image of a sample of vegetative B. thuringiensis cells, spores and GFP1Ab crystals. (B) GFP fluorescence image of a sample of vegetative B. thuringiensis cells, spores and GFP-Cry1Ab crystals. (C) Merger of (A) and (B) to show the fluorescent crystals and non-fluorescent spores and vegetative cells in the mixture. (D) Fluorescence from purified GFPCry1Ab crystals in Tris-EDTA buffer. (E) Fluorescent Crystals of mCherry 1Ab fusion protein. (F) Background (no fluorescence) sample of B. thuringiensis cells producing spores and crystals of Cry1Ab without any fusion protein.

FIG. 8: Schematic of the flow of crystals in the vascular system and localized fluorescence from GFP fused to the crystals.

FIG. 9: (A) Sequence data from an exemplary Ricin-Cry1Ab vector (B) Sequence data from an exemplary LcrV-Cry1Ab vector

FIG. 10: Sequence data from an exemplary ESAT6-Cry1Ab vector

FIG. 11: Flow chart of an exemplary method for growth, isolation & purification of crystals of fusion proteins used to generate an immune response.

FIG. 12: Western blot of protease treated: (A) Cry1Ab protein using anti-Cry antibody and (B) Ricin-Cry1Ab fusion protein using anti-Ricin antibody.

FIG. 13: SDS-PAGE gel showing successful purification of ESAT6 mutants

FIG. 14: Dot blots using anti-ESAT6 antibody to quantitate the amount of crosslinking.

FIG. 15: Dot blot using anti-ESAT6 antibody, 1=ESAT6 from M. marinum (fusion ESAT-1 Ab expression), 2=Buffer control (Phosphate buffer used for crosslinking), 3=ESAT6 from M. tuberculosis (crosslinked to Cry1Ab crystals), 4=ESAT6 protein from M. tuberculosis (control), 5=ESAT6 protein mixed with crystals of 1Ab (not crosslinked control), 6=crystals of Cry1Ab (crystal control)

FIG. 16: Western Blot using 1:10,000 dilution of anti LcrV antibody on LcrV-Cry1Ab fusion crystals solubilized in 50 mM Na₂CO₃ pH10.5 for 1 hr (lane 2) and 2 hrs (lane 4). Controls on lane 1 and 3 include Crystals of Cry1Ab solubilized for 1 hr and 2 hrs respectively.

FIG. 17: Graph showing Antibody responses in Balb/c mice toward ESAT6 and ESAT6-Cry1Ab crystals. Mice were immunized at 0, 2, 4 weeks with 10 μg/mouse of ESAT6-Cry crystals or 50 μg/mouse of purified recombinant ESAT6-TTP2/MVFP. Colorimetric ELISA assays were developed with serum diluted at 1:250 titer.

FIG. 18: 2-D schematic illustrating Cry crystal construction. (A) cry gene yields Cry crystal, (B) cry-sod gene fusion yields Cry-SOD crystal, (C) dual expression of cry-sod and cry-gpx yields Cry-SOD/GPx crystal.

FIG. 19: Images of fluorescent crystals under Nikon 80i microscope: (A) Fluorescence from purified GFP-Cry1Ab crystals in Tris-EDTA buffer. (B) Fluorescent Crystals of a mCherry-Cry1Ab fusion protein.

FIG. 20: Chemiluminescence of Cry-luciferase crystals treated with luciferin, together with the barely visible non-luminescent control.

FIG. 21: Effect of PEGylation on macrophage phagocytosis. (A) Cry-GFP crystals control; (B) PEGylated Cry-GFP crystals. The nuclei are stained with DAPI.

FIG. 22: Fluorescent micrographic images showing short-term uptake of Cry-GFP crystals by macrophages: (A) macrophages after 15 minutes; (B) macrophages after 4 hrs.

FIG. 23: Fluorescent micrographic images confirming that NIH3T3 fibroblasts engulf Cry3Aa-mCherry crystals (see red spots surrounding the DAPI stained nuclei).

### DETAILED DESCRIPTION

Unless otherwise defined, all technical and scientific terms used herein have the same meaning as commonly understood by one of ordinary skill in the art to which the embodiments pertain. Although methods and materials similar or equivalent to those described herein can be used in the practice or testing of various embodiments, suitable methods and materials are described below. All publications, patent applications, patents, and other references mentioned herein are incorporated by reference in their entirety for all purposes. In case of conflict, the present specification, including definitions, will control. In addition, the materials, methods, and examples are illustrative only and not intended to be limiting.

The section headings used herein are for organizational purposes only and are not to be construed as limiting the described subject matter in any way. It will be appreciated that there is an implied “about” prior to metrics such as temperatures, concentrations, and times discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention. The articles “a” and “an” are used herein to refer to one or to more than one (i.e., to at least one) of the grammatical object of the article. By way of example, “an element” means one element or more than one element.

An exemplary embodiment comprises a protein crystal that is generated in vivo in a cell. In preferred embodiments, the protein crystals are generated in a bacterium. In exemplary embodiments, the crystal proteins are produced by the gram positive bacterium, B. thuringiensis. Various other bacteria may also be used to produce the crystals in vivo, for example, Bacillus subtilis has been shown to produce heterologous Cry protein crystals. See Agaisse, H. and Lereclus, D. (1994) 176 (15) Journal of Bacteriology, p. 4734-4741. In alternative embodiments, the fusion protein crystals may be generated in vivo in Eukaryotic cells. For example, successful in vivo expression of Bt crystals in chloroplasts has been demonstrated in tobacco plants. See Cosa et al. (2001) Nature Biotechnology 19:71-74.

The term “Cry protein” or “Cry polypeptide” as used herein, refers to any one of the Cry polypeptides derived from Bacillus thuringiensis. A Cry protein, as used herein, can be a protein in the full length size, or can be in a truncated form as long as in vivo crystal forming activity is retained. The Cry protein can be a combination of different proteins in a hybrid or fusion protein. A “cry gene” or“cry DNA”, as used herein, is a DNA sequence encoding a Cry protein.

In various embodiments, the biologically synthesized crystals are fairly consistent in size with about 150-500 protein molecules per crystal in them depending on the crystal size. In Bacillus, the crystals may be generated during the sporulation phase of the bacterium and are formed alongside the spore in a bacterium. Cry proteins are harmless to humans and other mammals. Embodiments exploit Cry crystals as a common platform for a range of biological and industrial applications.

As can be appreciated, the Cry crystal fusion technology and/or the Cry protein crosslinking technology provides a platform for producing crystals displaying an almost limitless range of heterologous polypeptides. In the context of a Cry fusion protein, each individual heterologous protein may possess unique folding characteristics during crystal formation. The size of the pocket generated within the crystal may vary not only in its size, but also in its shape. Accordingly, it is difficult to specify an upper limit to the size of a protein that may be incorporated into a fusion crystal.

Embodiments include Cry polypeptides and Cry fusion polypeptides derived from Bacillus thuringiensis Cry polypeptides (e.g., Cry1Aa, Cry1Ab Cry2Aa, Cry3Aa, Cry4Aa, Cry4Ba, Cry11Aa, Cry11Ba, and Cry19Aa) including, but not limited to, the Cry-derived polypeptides of SEQ ID NOS: 2, 4, 6, 8, 10, 12, 14, 16, and 18. (See the attached Sequence Listing.) In addition to the polypeptide sequence of Cry-derived polypeptides, it will be appreciated that polypeptides also encompass variants thereof, including, but not limited to, any fragment, analog, homolog, naturally occurring allele, or mutant thereof. Polypeptides also encompass those polypeptides that are encoded by a Cry-derived nucleic acid. In various embodiments, shuffled polypeptides that form crystals in vivo and are at least 80%, 81%, 82%, 83%, 84%, 88%, 90%, 95%, 98%, 99% or 99.5% identical to the polypeptide sequence of any of SEQ ID NOS: 2, 4, 6, 8, 10, 12, 14, 16, and 18, or variants thereof. Methods of production of the polypeptides of the invention, e.g., by recombinant means, are also provided. Compositions comprising one or more polypeptides of the invention are also encompassed.

Embodiments include Cry-derived nucleic acid molecules of SEQ ID NOS: 1, 3, 5, 7, 9, 11, 13, 15, and 17. Also encompassed are fragments and analogs which encode polypeptides that are at least partially functionally active, i.e., they are capable of forming biologically synthesized crystals. In an embodiment, it encompasses an isolated shuffled nucleic acid molecule that is at least 90%, 91%, 92%, 93%, 94%, 95%, 96% 97%, 98%, 99% or 99.5% identical to any of SEQ ID NOS: 1, 3, 5, 7, 9, 11, 13, 15, and 17, or a compliment thereof. Vectors comprising nucleic acids of the invention are also encompassed. Cells or plants comprising embodied vectors are also encompassed.

In some embodiments, the protein crystals comprise fusion polypeptides comprising a Cry protein, or some truncated crystal-forming Cry protein component, fused to a heterologous polypeptide. In an exemplary embodiment, the fusion polypeptide is expressed and forms a crystal in vivo in a cell. In specific embodiments, the crystal of the fusion polypeptide is produced within a Bt cell. Additionally, various embodiments may be harvested directly from Bt cells. The fusion polypeptide crystals of an exemplary embodiment are stable. In exemplary embodiments, a simple purification strategy makes them relatively cheap to obtain. Various embodiments include agents and methods for diverse applications including, but not limited to, vaccines, imageable agents, molecular targeting agents, agents for delivering therapeutic enzymes and proteins to specific cells or tissues, blood substitutes, and agents for transport and delivery of biomolecules in animal models as well as in humans.

Exemplary embodiments are distinguishable from other protein crystals grown by standard methods of protein crystallographers. The crystals (e.g., Cry1Ab) of exemplary embodiments are preferably produced within a B. thuringiensis cell. As a result, biologically synthesized Cry crystals are fairly consistent in size, and can be directly harvested as a biomaterial via a remarkably simple purification strategy.

In various embodiments, fusion polypeptides including a Cry protein agent are provided. Nucleic acid sequences encoding various fusion polypeptides are also provided.

Various embodiments comprise a recombinant protein crystal. In exemplary embodiments, the crystal or crystals comprise a Cry polypeptide or a Cry fusion polypeptide. In some embodiments, at least one agent, polypeptide, nucleic acid, and or molecule may be bound and/or crosslinked to the crystal. As used herein, a “crystal” refers to is a solid material, whose constituent atoms, molecules, or ions are arranged in an orderly repeating pattern extending in all three spatial dimensions. The determination of a crystal can be determined by any means including, inter alia, optical microscopy, electron microscopy, x-ray powder diffraction, solid state nuclear magnetic resonance (NMR) or polarizing microscopy. Microscopy can be used to determine the crystal length, diameter, width, size and shape, as well as whether the crystal exists as a single particle or is polycrystalline.

The term “endospore” used herein refers to any spore that is produced within a bacterium during periods of environmental stress.

A “fusion polypeptide,” as used herein, is a polypeptide containing portions of amino acid sequence derived from two or more different proteins.

As used herein, the term “nucleic acid sequence” refers to a polymer of deoxyribonucleotides or ribonucleotides in the form of a separate fragment or as a component of a larger construct. Nucleic acids expressing the products of interest can be assembled from cDNA fragments or from oligonucleotides that provide a synthetic gene which is capable of being expressed in a recombinant transcriptional unit. Polynucleotide or nucleic acid sequences include DNA, RNA, and cDNA sequences.

Nucleic acid sequences utilized can be obtained by several methods. For example, the DNA can be isolated using hybridization procedures that are well known in the art. These include, but are not limited to: (1) hybridization of probes to genomic or cDNA libraries to detect shared nucleotide sequences; (2) antibody screening of expression libraries to detect shared structural features; and (3) synthesis by the polymerase chain reaction (PCR). Sequences for specific genes and polypeptides can also be found in GenBank, National Institutes of Health computer database. See http://www.ncbi.nlm.nih.gov/Genbank/

In another aspect, embodiments provide a method of producing a desired protein crystal comprising a fusion polypeptide, by growing host cells containing a nucleic acid encoding the fusion polypeptide under conditions that allow expression of the nucleic acid sequence, and recovering a crystal formed in the host cell. The nucleic acid sequences of various embodiments can be operably linked to a promoter for expression in a prokaryotic or eukaryotic expression system. For example, a nucleic acid can be incorporated in an expression vector.

Delivery of a nucleic acid can be achieved by introducing the nucleic acid into a cell using a variety of methods known to those of skill in the art. For example, the construct can be delivered into a cell using a colloidal dispersion system. Alternatively, nucleic acid construct can be incorporated (i.e., cloned) into an appropriate vector. For purposes of expression, the nucleic acid sequences encoding the fusion polypeptide may be inserted into a recombinant expression vector. The term “recombinant expression vector” refers to a plasmid, virus, or other vehicle known in the art that has been manipulated by insertion or incorporation of the nucleic acid sequences encoding the fusion polypeptides. The expression vector typically contains an origin of replication, a promoter, as well as specific genes that allow phenotypic selection of the transformed cells. Vectors suitable for use include, but are not limited to, the pSB6341 Ab expression vector for expression in Bacillus thuringiensis (Bt), the pHT315 expression vector for expression in Bacillus thuringiensis (Bt), the T7-based expression vector for expression in bacteria (Rosenberg et al., Gene, 56:125, 1987), the pMSXND expression vector for expression in mammalian cells (Lee and Nathans, J. Biol. Chem., 263:3521, 1988), baculovirus-derived vectors for expression in insect cells, cauliflower mosaic virus, CaMV, tobacco mosaic virus, TMV.

Depending on the vector utilized, any of a number of suitable transcription and translation elements, including constitutive and inducible promoters, transcription enhancer elements, transcription terminators, etc. may be used in the expression vector (see, e.g., Bitter et al., Methods in Enzymology, 153:516-544, 1987). These elements are well known to one of skill in the art.

The term “operably linked” or “operably associated” refers to functional linkage between the regulatory sequence and the nucleic acid sequence regulated by the regulatory sequence. The operably linked regulatory sequence controls the expression of the product expressed by the nucleic acid sequence. Alternatively, the functional linkage also includes an enhancer element.

“Promoter” means the minimal nucleotide sequence sufficient to direct transcription. Also included are those promoter elements that are sufficient to render promoter-dependent nucleic acid sequence expression controllable for cell-type specific, tissue specific, or inducible by external signals or agents; such elements may be located in the 5′ or 3′ regions of the native gene, or in the introns.

“Gene expression” or “nucleic acid sequence expression” means the process by which a nucleotide sequence undergoes successful transcription and translation such that detectable levels of the delivered nucleotide sequence are expressed in an amount and over a time period so that a functional biological effect is achieved.

An expression vector can be used to transform a target cell. By “transformation” is meant a permanent genetic change induced in a cell following incorporation of new DNA (i.e., DNA exogenous to the cell). Where the cell is a mammalian cell, the permanent genetic change is generally achieved by introduction of the DNA into the genome of the cell. By “transformed cell” is meant a cell into which (or into an ancestor of which) has been introduced, by means of recombinant DNA techniques, a DNA molecule encoding a fusion protein comprising a Cry protein, or a fragment thereof. Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

A crystal comprising a fusion polypeptide can be produced by expression of nucleic acid encoding the protein in prokaryotes. These include, but are not limited to, microorganisms, such as bacteria (e.g., Bt) transformed with recombinant plasmid DNA, bacteriophage DNA, or cosmid DNA expression vectors encoding a fusion protein. Vector constructs can be expressed in B. thuringiensis in large scale.

In various embodiments, purification of desired crystals is simple and cost effective. Initially, a shuttle vector is produced encoding a heterologous polypeptide fused to a Cry polypeptide, or a crystal forming fragment thereof. Preferably, the expression vector is optimized for overexpression in B. thuringiensis. The vector (e.g., pHT315-fusionCry1Ab) may be transformed into a bacterium (e.g., a B. thuringeinsis cell) where the fusion protein is produced and forms crystals within the cell. In various embodiments, desired crystals may be generated during the sporulation phase of the bacterium and are formed alongside the spore in the bacterium (e.g., B. thuringiensis cells). Subsequently the spores/crystal mixture may be released from autolyzed Bt. Density gradient centrifugation may be performed using a Renograffin gradient. The bands containing the spores and crystals may then be isolated. In the final step, the spore and crystal particles may be separated by CM-cellulose chromatography to generate purified crystals comprising the desired fusion polypeptide.

In alternative embodiments, Cry crystals purification from bacteria may also be accomplished when the expression sequences include tags for one-step purification such as by nickel-chelate chromatography. The construct can also contain a tag to simplify isolation of the fusion polypeptide. For example, a polyhistidine tag of, e.g., six histidine residues, can be incorporated at the amino terminal end of the fluorescent protein. The polyhistidine tag allows convenient isolation of the protein in a single step by nickel-chelate chromatography. Other possible tags include CBP, CYD (covalent yet dissociable NorpD peptide), Strep II, FLAG, HPC (heavy chain of protein C) peptide tags, and the GST and MBP protein fusion tag systems. The fusion polypeptide can also be engineered to contain a cleavage site to aid in protein recovery. Alternatively, the fusion polypeptides of the embodiments can be expressed directly in a desired host cell for application in situ.

In other embodiments, the crystals can be used in an unpurified or partially purified state from which the activity associated with the crystal properties can still be utilized. Examples include the use of the crystal-containing cells or lysed proteins obtained after cell growth, or the crystal-containing fraction generated following centrifugation.

When the host is a eukaryote, such methods of transfection of DNA as calcium phosphate co-precipitates, conventional mechanical procedures, such as microinjection, electroporation, insertion of a plasmid encased in liposomes, or virus vectors may be used. Eukaryotic cells can also be cotransfected with DNA sequences encoding the fusion polypeptide, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukaryotic Viral Vectors, Cold Spring Harbor Laboratory, Gluzman ed., 1982). Preferably, a eukaryotic host is utilized as the host cell, as described herein.

Eukaryotic systems, and preferably mammalian expression systems, allow for proper post-translational modifications of expressed mammalian proteins to occur. Eukaryotic cells that possess the cellular machinery for proper processing of the primary transcript, glycosylation, phosphorylation, and advantageous secretion of the gene product should be used as host cells for the expression of the polypeptide. Such host cell lines may include, but are not limited to, CHO, VERO, BHK, HeLa, COS, MDCK, Jurkat, HEK-293, and WI38.

Techniques for the isolation and purification of either microbially or eukaryotically expressed polypeptides may be by any conventional means, such as, for example, preparative chromatographic separations and immunological separations, such as those involving the use of monoclonal or polyclonal antibodies or antigen.

Pharmaceutical Compositions

In another aspect, compositions, e.g., pharmaceutically acceptable compositions, are provided which include desired crystals comprising either a fusion polypeptide, or other molecule, nucleic acid, or protein bound or crosslinked to cry protein crystals, as described herein, formulated together with a pharmaceutically acceptable carrier. For applications in animals, mucosal administration of the bacterial isolate or some partially purified crystalline fraction is also acceptable.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal oral, nasal, or epidermal administration (e.g., by injection or infusion).

The compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Useful compositions are in the form of injectable or infusible solutions. A useful mode of administration is parenteral (e.g., intravenous, subcutaneous, intraperitoneal, intramuscular). For example, the protein or crystal can be administered by intravenous infusion or injection. In another embodiment, the protein or crystal is administered by intramuscular or subcutaneous injection. These protein or crystal compositions can also be administered via oral or nasal administration. For example, when delivered intranasally (i.n.), Cry1Ac is a potent mucosal immunogen and adjuvant. See Rodriguez-Monroy (2010) Scand. J. of Immunology 71, pp: 159-168.

Compositions for administration to animals and humans typically should be stable under the conditions of manufacture and storage. The composition may be formulated as a solution, microemulsion, dispersion, liposome, or other ordered structure suitable to high crystal concentration, including the bacterial cell mass as isolated directly from cell culture or some lyophilized form. Sterile injectable solutions may be prepared by incorporating the active compound (e.g., Cry fusion polypeptide, Cry crystals crosslinked with heterologous molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin. For administration in animals, feeding the animals the cell paste, or a partially purified composition,—either directly, lyophilized, or in some dispersion may be advantageous.

The compositions can be administered by a variety of methods known in the art, although for many therapeutic and prophylactic applications. As will be appreciated by the skilled artisan, the route and/or mode of administration will vary depending upon the desired results.

In exemplary embodiments, a composition (e.g., crystals comprising a Cry fusion polypeptide, Cry crystals crosslinked with a heterologous molecule or agent) may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. Therapeutic compositions can be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a Cry fusion polypeptide or fragment thereof is 0.1-100 mg/kg, e.g., 1-10 mg/kg. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. The exact dosage can vary depending on the route of administration. For intramuscular injection, the dose range can be 100 μg (microgram) to 10 mg (milligram) per injection. Multiple injections may be needed.

The pharmaceutical compositions described herein can include a therapeutically effective amount or a prophylactically effective amount of a desired Cry crystal comprising a Cry fusion polypeptide and/or a Cry crystal (comprising either a Cry fusion polypeptide or a Cry polypeptide) crosslinked with heterologous molecule. A therapeutically effective amount of a desired Cry crystal comprising a Cry fusion polypeptide and/or a Cry crystal (comprising either a Cry fusion polypeptide or a Cry polypeptide) crosslinked with heterologous molecule varies according to factors such as the disease state, age, sex, and weight of the individual, and the ability of the composition to elicit a desired response in the individual. A therapeutically effective amount is also one in which any toxic or detrimental effects of the pharmaceutical composition is outweighed by the therapeutically beneficial effects. The ability of a compound to inhibit a measurable parameter can be evaluated in an animal model system predictive of efficacy in the target subject (e.g., a human subject). Alternatively, this property of a composition can be evaluated by examining the ability of the compound to modulate, such modulation in vitro by assays known to the skilled practitioner.

A “prophylactically effective amount” refers to an amount effective, at dosages and for periods of time necessary, to achieve the desired prophylactic result, e.g., protective immunity against a subsequent challenge by a pathogen. Typically, since a prophylactic dose is used in subjects prior to or at an earlier stage of disease, the prophylactically effective amount will be less than the therapeutically effective amount.

Also provided herein are kits including one or more of a nucleic acid vector encoding a Cry fusion polypeptide, or a crystal-forming component thereof, bacteria for in vivo expression of a desired protein crystal, and or a composition comprising a desired Cry crystal comprising a Cry fusion polypeptide, or a crystal-forming component thereof, and/or a Cry crystal (comprising either a Cry fusion polypeptide or a Cry polypeptide) crosslinked with a heterologous molecule or agent. The kit can include one or more other elements including: instructions for use; other reagents, e.g., a label, a therapeutic agent, or an agent useful for crosslinking, recominantly engineering or otherwise coupling or fusing a Cry protein to a therapeutic agent and or diagnostic agent; devices or other materials for preparing the composition for administration; pharmaceutically acceptable carriers; and devices or other materials for administration to a subject.

Instructions for use can include instructions for diagnostic applications of the protein crystals, polypeptides, nucleic acid sequence, in vitro, e.g., in a sample, e.g., a biopsy or cells from a patient, or in vivo. The instructions can include instructions for therapeutic or prophylactic application including suggested dosages and/or modes of administration.

The kit can further contain at least one additional reagent, such as a diagnostic or therapeutic agent, e.g., one or more additional desired crystals and/or an agent in one or more separate pharmaceutical preparations.

Therapeutic Uses

The new nucleic acids, fusion polypeptides, and crosslinked species described herein have in vitro and in vivo diagnostic, therapeutic, and prophylactic utilities. For example, the vaccines and fluorescent microdots may be administered to cells in culture, e.g., in vitro or ex vivo, or in a subject, e.g., in vivo, to treat, prevent, and/or diagnose various diseases.

As used herein, the term “subject” is intended to include humans and non-human animals. The term “non-human animals” includes all vertebrates, e.g., mammals and non-mammals, such as non-human primates, pigs, chickens and other birds, mice, dogs, cats, cows, horses, and fish.

Methods of administering a Cry crystal comprising a fusion polypeptide and/or a Cry crystal crosslinked with a heterologous molecule and or agent are described above. Suitable dosages of the molecules used will depend on the age and weight of the subject and the particular composition used. The described vaccines can be used to prevent various disease conditions by inducing a protective immunity in the inoculated subject, or to treat an existing disease state if improved immune responses can be useful in controlling the relevant pathogen. For example, the Cry crystals comprising fusion proteins comprising Cry polypeptides fused with specific antigens can be used to prevent, reduce, or alleviate bacterial and or an acute influenza infection.

In other embodiments, immunogenic compositions and vaccines that contain an immunogenically effective amount of an antigenic polypeptide, or antigenic fragments thereof, fused or crosslinked to a Cry crystal, are provided. Immunogenic epitopes in a polypeptide sequence can be identified according to methods known in the art, and proteins or fragments containing those epitopes can be delivered by various means, in a vaccine composition.

Suitable compositions can include, for example, lipopeptides (e.g., Vitiello et al., J. Clin. Invest., 95:341, 1995), peptide compositions encapsulated in poly(DL-lactide-co-glycolide) (“PLG”) microspheres (see, e.g., Eldridge et al., Molec. Immunol., 28:287-94, 1991; Alonso et al., Vaccine, 12:299-306, 1994; Jones et al., Vaccine, 13:675-81, 1995), peptide compositions contained in immune stimulating complexes (ISCOMS) (see, e.g., Takahashi et al., Nature, 344:873-75, 1990; Hu et al., Clin. Exp. Immunol., 113:235-43, 1998), and multiple antigen peptide systems (MAPs) (see, e.g., Tam, Proc. Natl. Acad. Sci. U.S.A., 85:5409-13, 1988; Tam, J. Immunol. Methods, 196:17-32, 1996). Toxin-targeted delivery technologies, also known as receptor-mediated targeting, such as those of Avant Immunotherapeutics, Inc. (Needham, Mass.) can also be used.

Useful carriers that can be used with immunogenic compositions and vaccines are well known, and include, for example, thyroglobulin, albumins such as human serum albumin, tetanus toxoid, polyamino acids such as poly L-lysine, poly L-glutamic acid, influenza, hepatitis B virus core protein, and the like. The compositions and vaccines can contain a physiologically tolerable (i.e., acceptable) diluent such as water, or saline, typically phosphate buffered saline. Besides the crystal itself, the compositions and vaccines may also include an additional adjuvant. Adjuvants such as incomplete Freund's adjuvant, aluminum phosphate, aluminum hydroxide, or alum are examples of materials well known in the art. Additionally, CTL responses can be primed by conjugating influenza or other viral polypeptides (or fragments, derivatives or analogs thereof) to lipids, such as tripalmitoyl-S-glycerylcysteinyl-seryl-serine.

Immunization with a composition or vaccine containing a protein composition, e.g., via injection, aerosol, oral, transdermal, transmucosal, intrapleural, intrathecal, or other suitable routes, induces the immune system of the host to respond to the composition or vaccine by producing large amounts of CTLs, and/or antibodies specific for the desired antigen. Consequently, the host typically becomes at least partially immune to later infection (e.g., M. tuberculosis), or at least partially resistant to developing an ongoing chronic infection, or derives at least some therapeutic benefit. For example, the subject is protected against subsequent infection by the target virus or bacteria.

The nucleic acid molecules are not limited strictly to molecules provided, including those set forth in the attached sequence listing. Rather, specific embodiments encompasses nucleic acid molecules carrying modifications such as substitutions, deletions, insertions, or inversions, which nevertheless encode proteins having substantially the crystal forming ability of the polypeptide according to the specific embodiments, and/or which can serve as hybridization probes for identifying a nucleic acid with one of the disclosed sequences. Included are nucleic acid molecules, the nucleotide sequence of which there is a portion that is at least 75% identical (e.g., at least 75%, 85%, 95%, or 99% identical) to the provided nucleotide sequences.

The determination of percent identity or homology between two sequences is accomplished using the algorithm of Karlin and Altschul (1990) Proc. Nat'l Acad. Sci. USA 87: 2264-2268, modified as in Karlin and Altschul (1993) Proc. Nat'l Acad. Sci. USA 90:5873-5877. Such an algorithm is incorporated into the NBLAST and XBLAST programs of Altschul et al. (1990) J. Mol. Biol. 215:403-410. BLAST nucleotide searches are performed with the NBLAST program, score=100, wordlength=12 to obtain nucleotide sequences homologous to the nucleic acid molecules. BLAST protein searches are performed with the XBLAST program, score=50, wordlength=3 to obtain amino acid sequences homologous to the protein molecules. To obtain gapped alignments for comparison purposes, Gapped BLAST is utilized as described in Altschul et al. (1997) Nucleic Acids Res. 25: 3389-3402. When utilizing BLAST and Gapped BLAST programs, the default parameters of the respective programs (e.g., XBLAST and NBLAST) are used. See http://www.ncbi.nlm.nih.gov.

Embodiments also include an isolated polypeptide encoded by a nucleic acid of an exemplary embodiment. An “isolated” polypeptide is a polypeptide that is substantially free from the proteins and other naturally occurring organic molecules with which it is naturally associated. Purity can be measured by any art-known method, e.g., column chromatography, polyacrylamide gel electrophoresis, or HPLC. An isolated polypeptide may be obtained, for example, by extraction from a natural source (e.g., a human cell); by expression of a recombinant nucleic acid encoding the polypeptide; or by chemical synthesis of the polypeptide. In the context of a polypeptide obtained by extraction from a natural source, “substantially free” means that the polypeptide constitutes at least 30% (e.g., at least 35%, 45%, 85%, etc.) of the dry weight of the preparation. A protein that is chemically synthesized or produced from a source different from the source from which the protein naturally originates, is by definition substantially free from its naturally associated components. Thus, an isolated polypeptide includes recombinant polypeptides synthesized, for example, in vivo, e.g., in Bt cells, or in vitro, e.g., in a mammalian cell line, E. coli or another single-celled microorganism, or in insect cells.

In exemplary embodiments, a Cry-heterologous polypeptide fusion vector may be transformed into B. thuringeinsis cells where the fusion protein is expressed. Crystals comprising the fusion protein may be generated during the sporulation phase of the bacterium and are formed along with the spore in a bacterium, the macroscopic spore and crystal may be released allowing for their separation by centrifugation (e.g., in a renografin solution). In the final step, the spore and crystal particles may be separated by chromatography (e.g., CM-cellulose type).

In various embodiments, the endospores may be used as a convenient storage vehicle for transporting and packaging Bt cells transformed with nucleic acids encoding fusion polypeptides. Endospores are resistant to desiccation, temperature, starvation, and other environmental stresses. Accordingly, endospores containing nucleic acids encoding fusion polypeptides may be easily packaged and transported. When desired, the endospores of various embodiments may be reactivated through a process that includes the steps of activation, germination, and outgrowth to develop into a fully functional vegetative bacterial cell. These bacterial cells may then form crystals comprising fusion polypeptides.

In various embodiments, the polypeptides comprise an amino acid sequence, or a fragment thereof, of the sequences set forth (or provided by accession number). However, polypeptides of the exemplary embodiments are not limited to those having an amino acid sequence identical to one of sequences set forth (or provided by accession number). Rather, embodiments also encompasses conservative variants of the disclosed sequences. “Conservative variants” include substitutions within the following groups: glycine and alanine; valine, alanine, isoleucine, and leucine; aspartic acid and glutamic acid; asparagine, glutamine, serine, and threonine; lysine, arginine, and histidine; and phenylalanine and tyrosine.

Also included in the embodiments are polypeptides carrying modifications such as substitutions, deletions, insertions, or inversions, which polypeptides nevertheless have substantially the crystal forming ability of the Cry polypeptide. Consequently, included in the embodiments is a polypeptide, or a crystal forming fragment thereof, the amino acid sequence of which is at least 60% identical (e.g., at least 60%, 70%, 80%, or 95% identical) to an amino acid sequence set forth in the sequence information. “Percent identity” is defined in accordance with the algorithm described above.

### EXAMPLES

The following examples are included to demonstrate embodiments. It should be appreciated by those skilled in the art that the techniques disclosed in the examples which follow represent techniques discovered by the inventors to function well in practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention.

Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturers specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques of described herein are those well known and commonly used in the art.

(a) Imageable Agents

Monitoring of cellular events including heart conditions using tracer molecules, especially radionuclides have been established for more than a decade. However there exist potential risk factors associated with the use of radioisotopes to human health such as decrease in sperm count and the dangers of radiation expose in case of pregnant mothers. Embodiments include micromolecular bioprobes that may be used as alternative tracer molecules.

Exemplary embodiments comprise imageable crystals comprising fusion proteins comprising a fluorescent protein fused with a Cry polypeptide. In exemplary embodiments, the fusion polypeptides form Cry fusion protein crystals when the proteins are expressed within a bacterium. In at least one embodiment, the fluorescent protein is the green fluorescent protein (GFP) from Aequorea victoria. The chromophore for the protein is formed from the self-catalyzed coupling of serine, tyrosine and glycine amino acids in the core of the β-barrel protein. The non-toxic nature of these proteins in cellular environments, their facile coupling to exogenous genes, and multiple colors that can be obtained has led to their general application for in vivo studies.

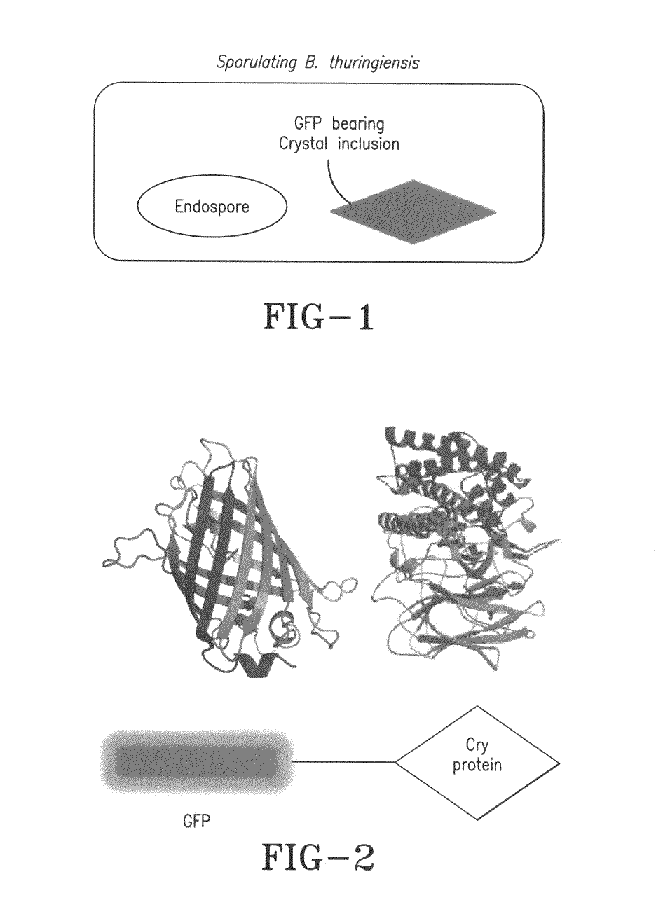

Various embodiments provide imageable agents by exploiting the ability of Bacillus thuringiensis (Bt) to form crystals of Cry proteins. In nature, Cry proteins protect the plants that Bt grows on by killing insect pests. When insects eat the plants, they also ingest the bacteria. Digestion of the bacteria releases the crystal and ultimately, the Cry protein that inserts into the insect midgut membrane and forms pores that ultimately kill the insect. Various embodiments utilize the unusual Cry protein crystals as the framework of a novel biomaterial (FIG. 1)—namely a biodegradable fluorescent microdot for use in various biomedical applications including intra-arterial monitoring of fluorescence in blood for quick and easy detection of blood flow in heart valves.

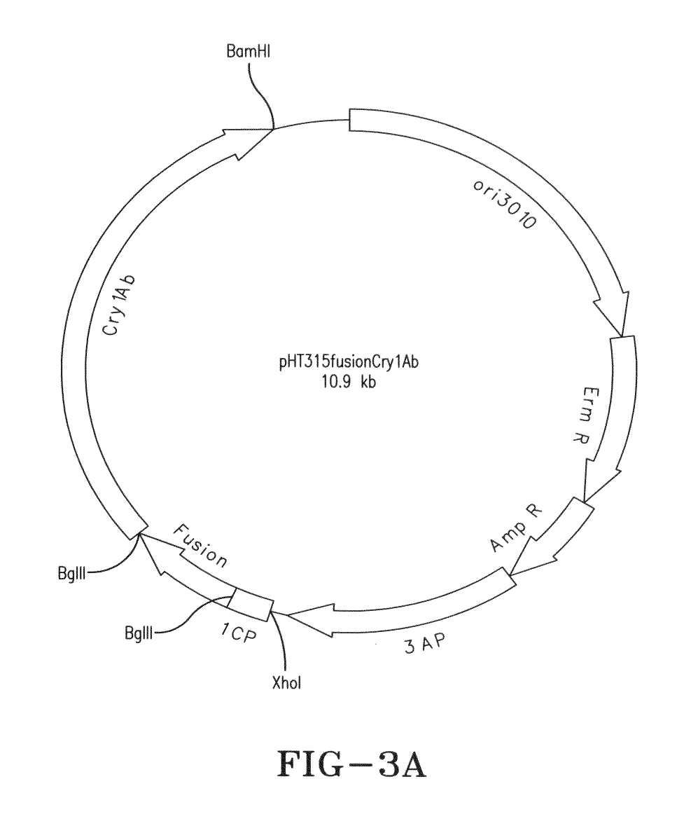

Accordingly, embodiments provide a GFP-Cry fusion protein (FIG. 2) that forms crystalline inclusions like isolated Cry protein, but now is highly fluorescent due to the GFP domain. In an exemplary embodiment, the “GFP-microdots” may have many GFP molecules spaced within the Cry protein framework and thus will exhibit much greater fluorescence than a single GFP molecule. Also like GFP molecules they are derived from, the proposed GFP-microdots could be made to absorb and fluoresce with a wide variety of wavelengths.

Using the fluorescent microdots from an exemplary embodiment, the rate of blood flow can be measured with confocal laser scanning microscopy (CLSM), used to generate three dimensional image constructions of complex biological specimens. Alternatively, Optical Coherence Tomography (OCT) may enable live imaging of sub-surface and inner opaque tissues by using interferometric principles. The OCT method separates scattered light and extracts out only the coherent or “in-phase” light to allow the generation of a sharper image. It is anticipated that most conventional fluorescence imaging techniques may be used to monitor the fluorescent crystals in the vascular system to obtain in depth images. Other similar studies have obtained images using an SPY imaging system (Novadaq Technologies, Canada) on pancreatic tissues. Fluorescent images using the SPY system on the heart have been shown. However, those systems utilize synthetic dyes. In contrast, the GFP-microdots of exemplary embodiments may be comprised of a fully biological material.

While generally stable, exemplary embodiments can be expected to be sensitive to proteases upon treatment, thereby enabling their eventual breakdown to amino acids once the imaging process is completed. However, various embodiments may possess site-directed mutations that cause the protein to degrade more slowly. For example, the C-terminus of the Cry1Ab proteins is known to be sensitive to trypsin and thus modifications may be made in the sequence of this region to reduce the degradation rate without substantially affecting the ability to form crystals.

In specific embodiments, a recognition domain or receptor may be added. Such a domain may be used to direct the dye to specific targets in the body. One specific example comprises the addition of a tumor associated protein called CD117, a marker of tumor blood vessels that, when displayed on these crystals (either by recombinant addition or by crosslinking the recognition domain to the crystals), could be used to direct the GFP-microdots to identify cancerous cells in the vascular system. In other embodiments, the imaging agent may be bound or crosslinked to a molecular targeting agent. In various embodiments, suitable molecular targeting agents include peptides, lectins, antibodies (monoclonal and polyclonal), aptamers, avimers, etc. In exemplary embodiments, the molecular targeting agent selectively binds a marker associated a disease condition occurring in a tissue.

(i) Construction of a GFP-Microdots

Cry proteins (e.g. Cry1Ab), when fused recombinantly with fluorescent partners (e.g. GFP, mCherry, etc.), express the protein in specific competent Bt cells that are directed by the bacterium to form a crystal. These fluorescent fusion crystals called microdots may be released into the media by the autolysis of the bacterium. Isolation and purification of the microdots maybe performed by a simple two step process of density gradient centrifugation using contrast enhancing solution followed by a pH gradient purification in a carboxymethyl Cellulose column to separate spores from fluorescent microdot crystals.

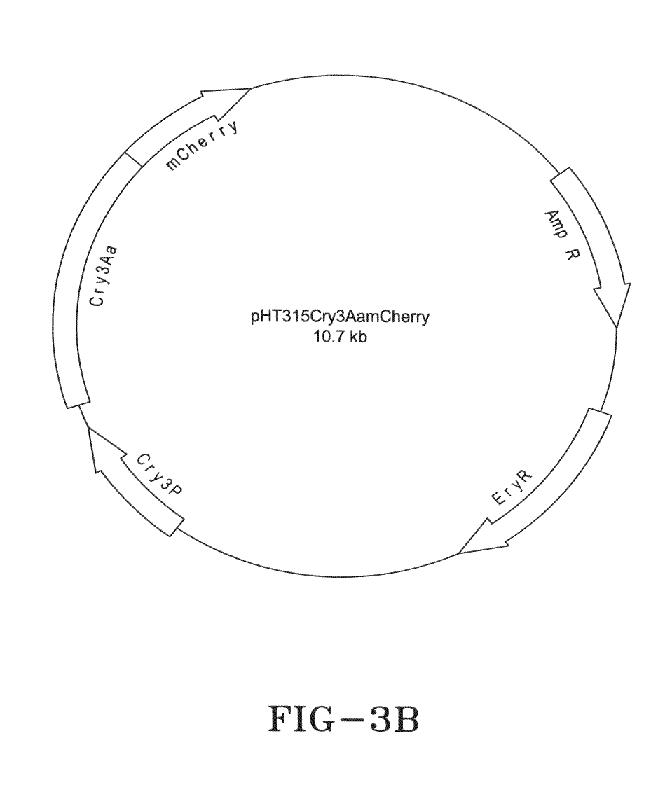

Molecular cloning of genes of different GFP mutants with fluorescence in green and red region of the visible spectrum were recombinantly engineered into a shuttle plasmid vector. FIGS. 3A-3C are three exemplary vectors for expressing Cry fusion protein crystals. FIG. 3A is a general shuttle vector for expressing Cry fusion crystals. The fusion protein expression vector in FIG. 3A comprises uses a pHT315 shuttle vector as a platform for expressing Cry fusion protein crystals. The cloning vector pHT315 is a “shuttle” vectors that was previously developed in order to carry out recombinant expression of crystal proteins of Bacillus thuringiensis. The vector bears an origin of replication (ori) for replicating in E. coli and another for Bt. In addition, the vector has two antibiotic resistance genes one for ampicillin (Amp^{R}) and second for Erythromycin (Ery^{R}). Detailed information about the construction of the pHT315 vector and for information about obtaining the vector, see Arantes, O. and Lereclus, D. Construction of cloning vectors for Bacillus thuringiensis, Gene 1991 v. 108; pp: 115-119. The pHT315 vector used as the shuttle vector in the examples was obtained from the laboratory of Didier Lereclus at the Institute Pasteur, Paris, France. Referring again to FIG. 3A, the vector comprises a sequence encoding a Cry1 promoter operably linked to the desired fusion polypeptide. The fusion polypeptide comprises a sequence encoding the desired heterologous peptide (e.g., GFP (SEQ ID NO: 19)) positioned upstream of a desired crystal forming Cry polypeptide, or crystal forming fragment thereof. Accordingly, the resulting peptide is a heterologous protein-Cry fusion peptide. In the exemplary embodiment shown in FIG. 3A, the Cry1Ab coding sequence (SEQ ID NO: 3) has been cloned into the exemplary shuttle vector.

The pHT315 vector, or analogous vectors, may be used to express a wide variety of Cry fusion crystals in vivo. In FIG. 3B, the pHT315 vector was used to create a Cry3Aa-mCherry fusion crystal expression vector. In this example, the Cry fusion protein vector is driven by a cry3A promoter (SEQ ID NO: 32) operably linked to a 1950 by cry3Aa coding sequence (SEQ ID NO: 7) from Bacillus thuringienis var tenebrionis. As shown in FIG. 3B, the gene for the fluorophore, mCherry (SEQ ID NO: 20) is positioned downstream of the cry3Aa gene followed by a stop codon. As can be appreciated, many other heterologous polypeptides may be fused to the Cry platform.

While the pHT315 is suitable for expressing Cry-heterologous protein fusion vectors, many other vectors are possible. A map of an alternative expression vector, pSB-GFP-1Ab, is shown in FIG. 3(C).

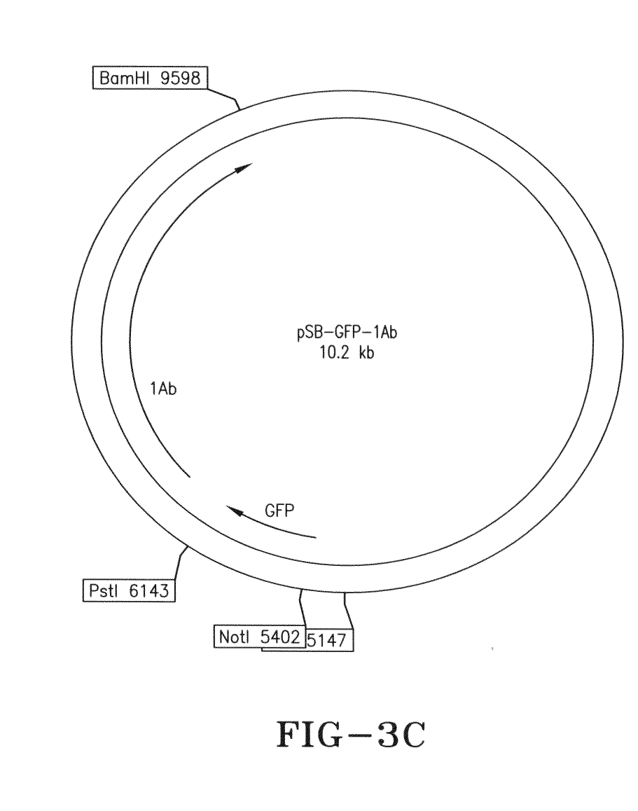

In one example, a nucleic acid encoding GFP (SEQ ID NO: 19) and a nucleic acid encoding Cry1Ab (SEQ ID NO: 3) was cloned into the pHT315 shuttle vector. FIGS. 4A and 4B show nucleic acid sequence data confirming correct incorporation of the nucleic acid sequence encoding GFP (SEQ ID NO: 19) (see FIG. 4A) and the sequence encoding Cry1Ab (SEQ ID NO: 3) (see confirmation in FIGS. 4A and 4B). In this example expression vector, the GFP sequence (SEQ ID NO: 19) is positioned upstream of the Cry1Ab sequence. In an exemplary embodiment, a short sequence encoding a linker region may be used to link the components of the fusion polypeptide. In alternative embodiments, various other Cry proteins (e.g., Cry3Aa may be fused to various other fluorescent proteins (e.g., mCherry (SEQ ID NO: 20)).

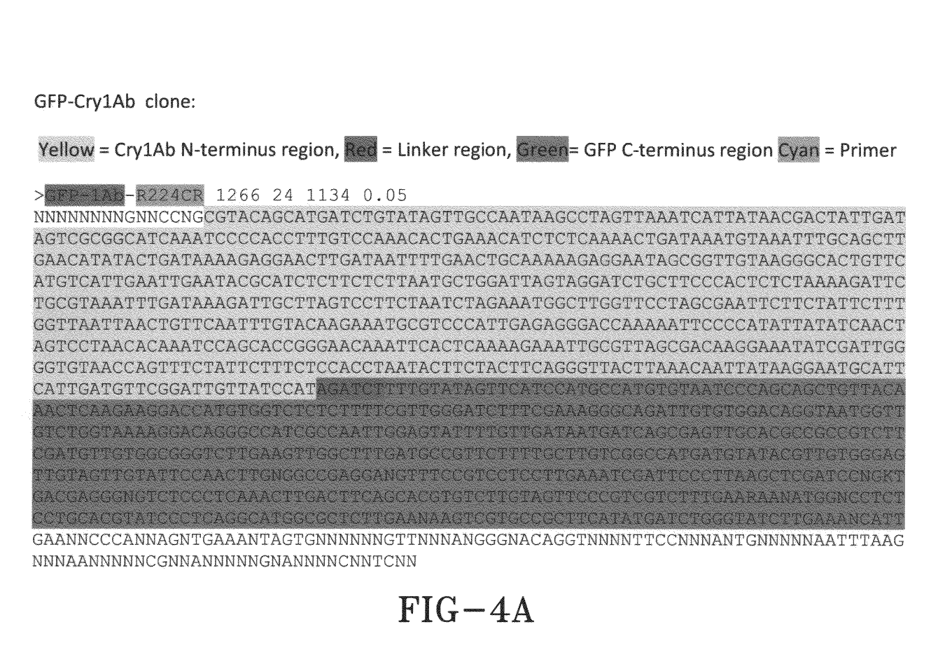

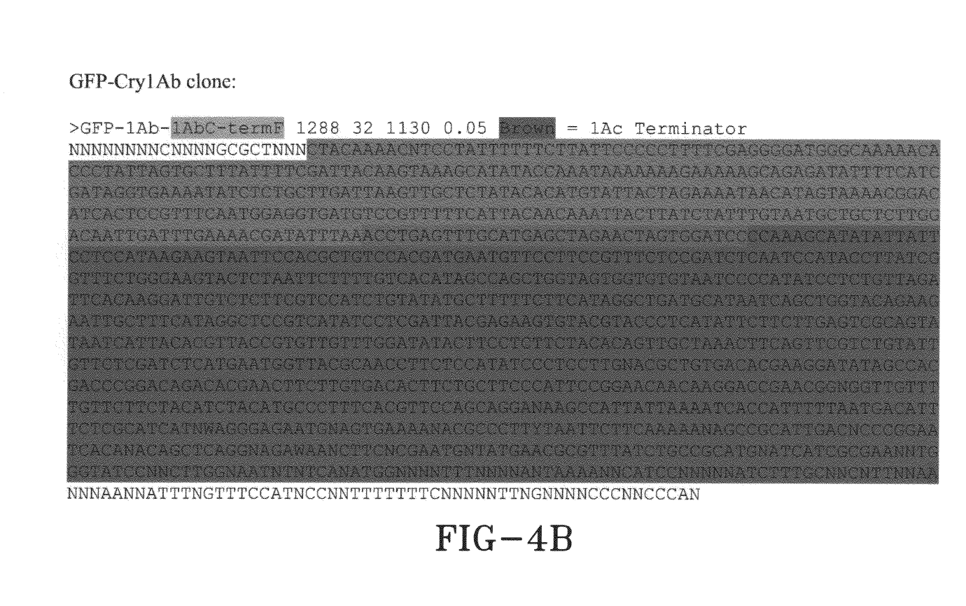

The fusion proteins of exemplary embodiments may be expressed in Bt to generate crystals during the sporulation phase of the bacterium. These crystals may be isolated using an established procedure like that shown schematically in FIG. 5. A SDS-PAGE gel of protein from Cry1Ab crystals obtained by this method is shown in FIG. 6. Referring to FIG. 6, Lane 1 was loaded with Purified Cry1Ab crystals; Lane 2 is a Control lane; Lane 3 was loaded with MW marker.

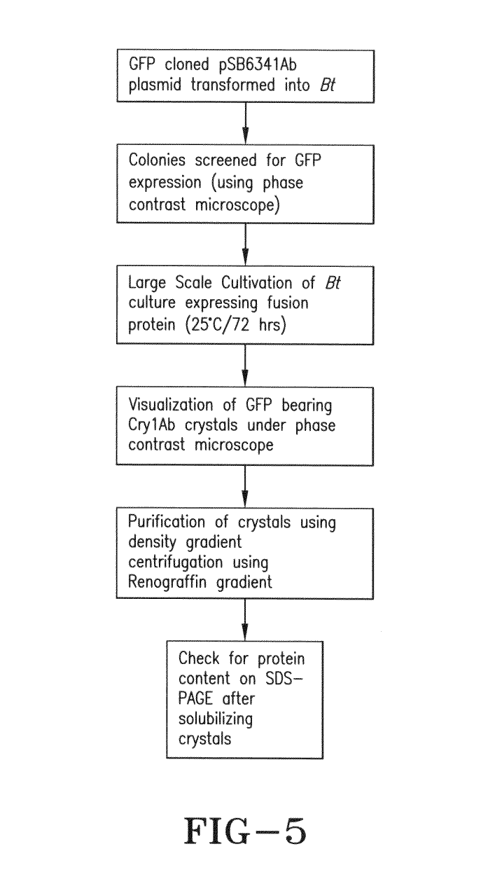

Exemplary embodiments use the above strategy to generate biological crystals comprising a Cry polypeptide (e.g., Cry1Ab) fused to a fluorescent protein (e.g., GFP and mCherry). FIG. 7 contains images of fluorescent crystals under Nikon 80i microscope: (A) Phase contrast image of a sample of vegetative B. thuringiensis cells, spores and GFP1Ab crystals. (B) GFP fluorescence image of a sample of vegetative B. thuringiensis cells, spores and GFP-Cry1Ab crystals. (C) Merger of (A) and (B) to show the fluorescent crystals and non-fluorescent spores and vegetative cells in the mixture. (D) Fluorescence from purified GFPCry1Ab crystals in Tris-EDTA buffer. (E) Fluorescent Crystals of mCherry 1Ab fusion protein. (F) Background (no fluorescence) sample of B. thuringiensis cells producing spores and crystals of Cry1Ab without any fusion protein.

As shown in the fluorescent confocal microscope images (FIG. 7), GFP-Cry1Ab and mCherry-Cry1Ab fusion proteins expressed in B. thuringiensis still produced biological crystals that are of similar size and shape. Notably, because GFP is only fluorescent when properly folded, its observed fluorescence demonstrates that the protein fold of GFP is retained in the crystal. Therefore, various other heterologous polypeptides, including enzymes and other biological proteins, should retain proper folding as well.

(ii) Application of GFP-Microdots

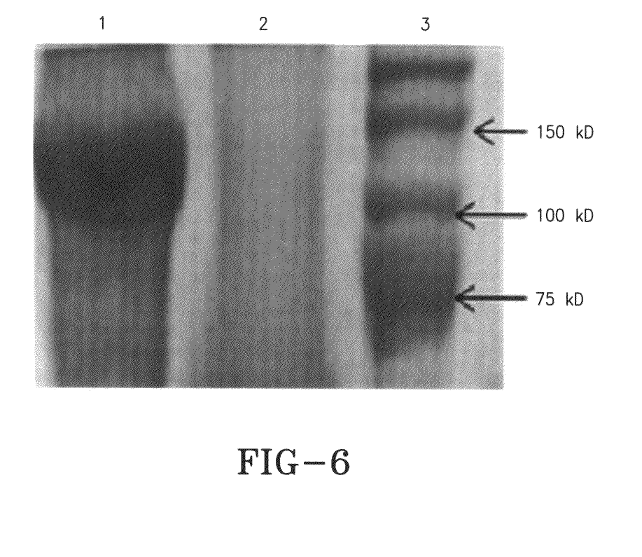

Various embodiments may be useful as a biodegradable imaging dye. Notably, because of the high GFP density present on Cry fusion protein crystals, visualization of the fluorescent molecule would be successful when injected at low microdot concentrations. A simplified schematic of the visualization in human vascular system is depicted in FIG. 8. In various embodiments, a subject would be injected with GFP-microdots. Visualization of the fluorescent light may be accomplished by focusing a beam of blue light to observe the heart and cardiovascular system. In alternative embodiments, imageable crystals may be ingested and followed through the digestive tract and may be useful in imaging, for example, the colon.

(b) Cry Crystal Species for Eliciting an Immune Response in a Subject

Embodiments overcome the toxicity and other adverse properties of traditional vaccine adjuvants by creating new cost effective strategies for application to a wide array of rare diseases. As with the GFP microdots discussed above, the adjuvants of exemplary embodiments rely on proteins that form crystals in vivo. In some embodiments, the crystals comprise a fusion polypeptide comprising a Cry protein fused to a heterologous polypeptide antigen. In other embodiments, the protein crystal serves as a platform for crosslinking or binding an antigenic protein, epitope, or molecule. The effectiveness of using protein crystals as vaccine adjuvants has been explored previously for crosslinked protein crystals (CLPC) of human serum albumin (HSA). Notably, the immunogenicity of the crystallized HSA was found to be higher than the soluble form of HSA. The improved immunogenicity stemmed from the better ability of crystallized HSA to enhance both the humoral mediated response and also enhanced the cell-mediated immune response. Presumably this is related to the crystals macroscopic size, longer lifetime, and multiple antigen molecules. One weakness of the past techniques, however, is that it was difficult to obtain uniform crystals (as one would want for application as a biological adjuvant) using standard in vitro protein crystallization approaches. In addition, there is no common and assured condition for crystallizing a protein in vitro. Thus the crystallization conditions of each antigen would have to be screened, as would the appropriate crosslinker and crosslinking conditions.

As demonstrated above, the ability of GFP-Cry1Ab fusion proteins to form crystals in vivo is a significant discovery. It is important evidence that the Cry1Ab protoxin protein also folds properly—most notably its cysteine-rich C-terminus, a region believed to be significant for crystal formation. It is currently thought that ionic and disulfide bonds formed by the protoxin C-terminal region accounts for crystal formation. This also means that the interactions that impart biological Cry toxin crystals with remarkable stability may still be retained—the typical conditions for solubilization requiring denaturants, proteases, and high pH. This stability suggests that the exemplary crystals should be able to withstand degradation inside the human serum or mucosal environment longer than the antigen itself, thereby providing for as a stable adjuvant that would serve in boosting the response to the antigen for longer periods of time and with fewer booster doses. Notably, as the proteins on the surface of the crystal were lost by slow degradation, the inner layers with a new set of antigens would be exposed.

Embodiments include a method for displaying antigens on a Cry crystal. Various embodiments comprise a plasmid vector encoding a Cry protein-antigen, crystal-forming protein. Embodied vectors may be constructed as described above for the imageable agents. Accordingly, at least one embodiment utilizes a pHT315 E. coli-B. thuringiensis shuttle vector containing the target antigen fused to the N-terminal domain of a Cry1Ab gene and which is optimized for overexpression in B. thuringiensis (FIG. 3B).

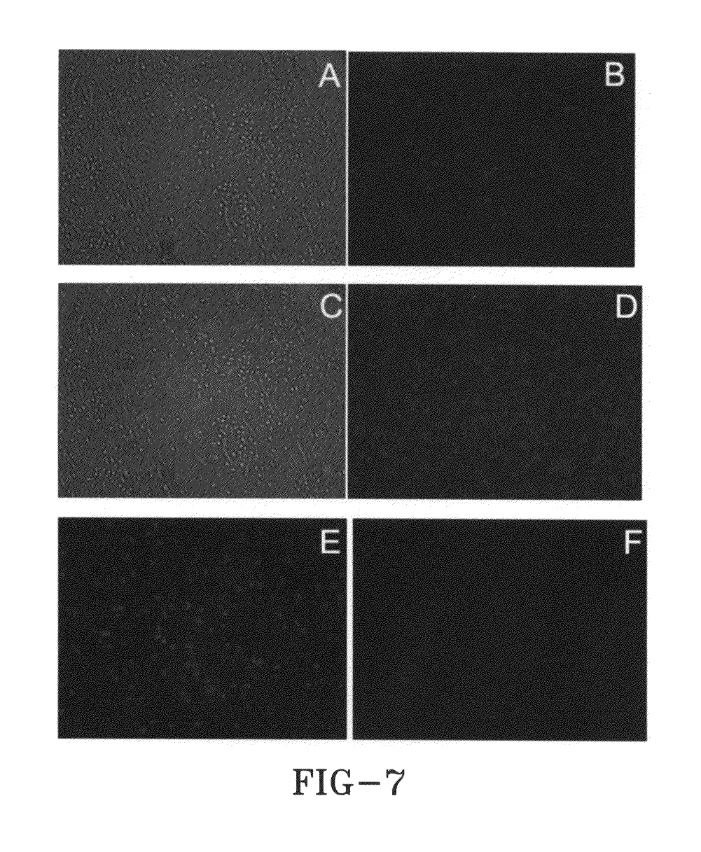

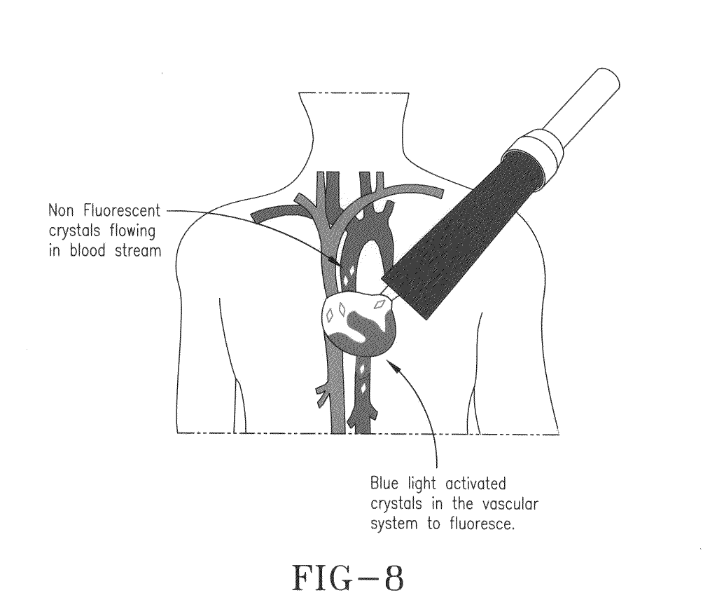

FIGS. 9 and 10 present sequence data confirming correct construction of plasmids encoding comprising Cry protein-antigen crystal-forming fusion polypeptides. FIG. 9A shows the sequence of one embodiment in which a nucleic acid encoding a Ricin B subunit (antigen) (SEQ ID NO: 21) has been fused (via a linker peptide) to the N-terminus of Cry1Ab. FIG. 9B shows sequence data confirming one embodiment in which a Cry1C promoter is operably linked to a sequence encoding the plaque LcrV antigen (SEQ ID NO: 22) fused to the N-terminus of Cry1Ab (SEQ ID NO: 3) (Cry portion not shown in FIG. 9B). LcrV is the V antigen of Yersinia pestis. In the same pHT315 vector, FIG. 10 shows the sequence of one embodiment in which a nucleic acid sequence encoding the ESAT6 antigen (SEQ ID NO: 23) was fused to a sequence encoding Cry1Ab (SEQ ID NO: 3). As shown in FIG. 10, a linker region may be used to link the components of the Cry fusion polypeptide.

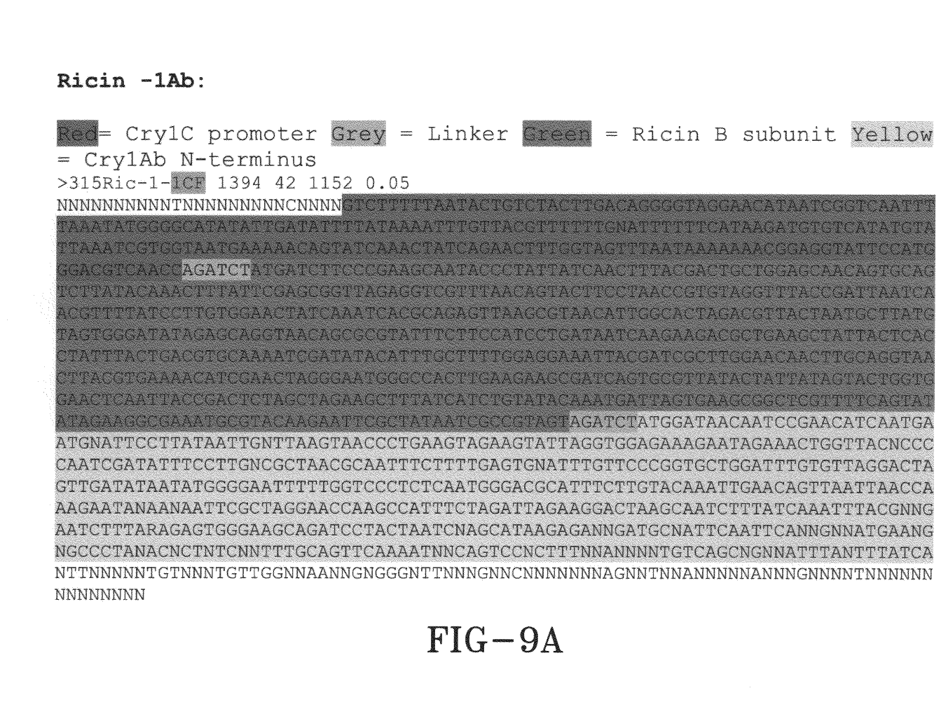

Cry protein-antigen, crystal-forming fusion polypeptides are easily and inexpensively produced and purified using methods contained herein. Referring to FIG. 11, in exemplary embodiments, the pHT315-fusion Cry1Ab vector may be transformed into B. thuringeinsis cells where the antigen-Cry1Ab fusion protein is produced (FIG. 11). After allowing the B. thuringiensis cells, the macroscopic spore and crystal may be released allowing for their separation by renograffin centrifugation. In the final step, the spore and crystal particles may be separated by CM-cellulose chromatography.

Embodiments also comprise other antigen-Cry toxin fusion proteins associated with mycobacterial diseases and visceral leishmaniasis. More specifically, various embodiments comprise a Cry protein crystal incorporating one of the following Mycobacterium antigens: fbpA, fbpB, fbpC, ESAT6, erp (pirG), Rv1477, (also MPT53, OmpAtb, IiA, p60, MPT53, OspA). Accordingly, various embodiments comprise vectors with a nucleic acid sequence encoding fbpA (SEQ ID NO: 24), fbpB (SEQ ID NO: 25), fbpC (SEQ ID NO: 26), ESAT6 (SEQ ID NO: 23), erp (pirG) (SEQ ID NO: 27), Rv1477 (SEQ ID NO: 28) fused to a Cry protein or crystal forming fragment thereof. Leishmania antigens include Leishmania A2 as well as the Leishmania antigen.

Embodiments may also include therapeutic compositions for eliciting an immune response in non-human animals. For example, Infectious Salmon Anemia Virus (ISAV) is a highly infectious disease of Atlantic salmon (Salmo salar). Embodiments include an expression vector encoding a Cry protein crystal fused (or alternatively crosslined) to a heterologous sequence encoding the M1 proton channel from ISAV (SEQ ID NO: 29).

(i) Ricin-Cry Crystals

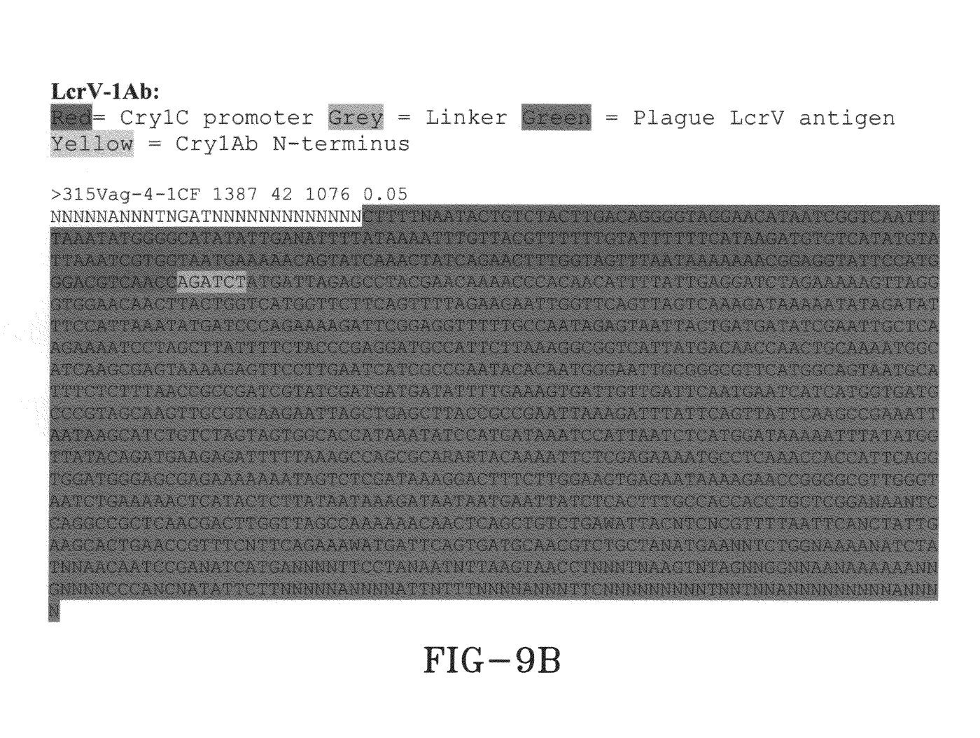

Referring to FIGS. 12A and B, a non-toxic fragment of subunit A of ricin antigen was fused to Cry1Ab and the fusion protein was expressed in B. thuringiensis. The resulting protein was activated with trypsin to test for protease resistance of the fusion protein. Using an anti-Ricin antibody, we were able to show that when the fusion protein crystals were solubilized at pH 10.5 and trypsin treated for 30 min, a 80-85 kDa band of the fusion protein was seen intact (20 kDa ricin fragment+65 kDa Cry1A toxin) on the blot (FIG. 12B) compared to a 65 kDa toxin only band of protease treated Cry toxin without fusion proteins (FIG. 12A) using anti-Cry antibody. Notably, these results confirm that the putative Ricin-Cry1Ab fusion protein crystals do indeed contain the Ricin fragment.

(ii) Preparation of ESAT6 Crosslinked to Crystals of Cry1Ab

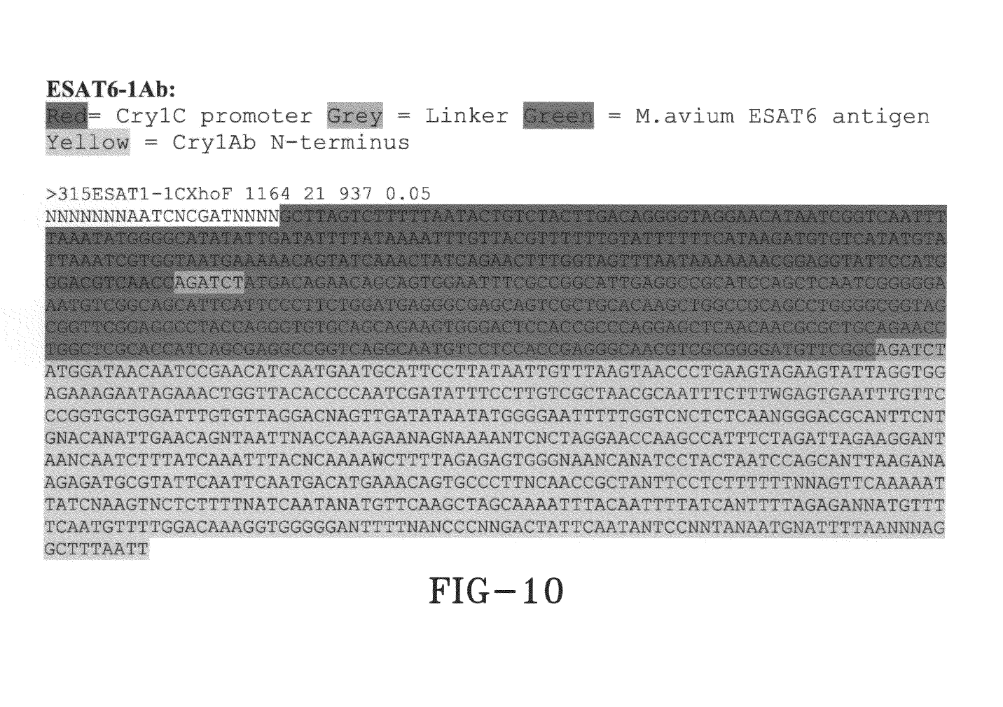

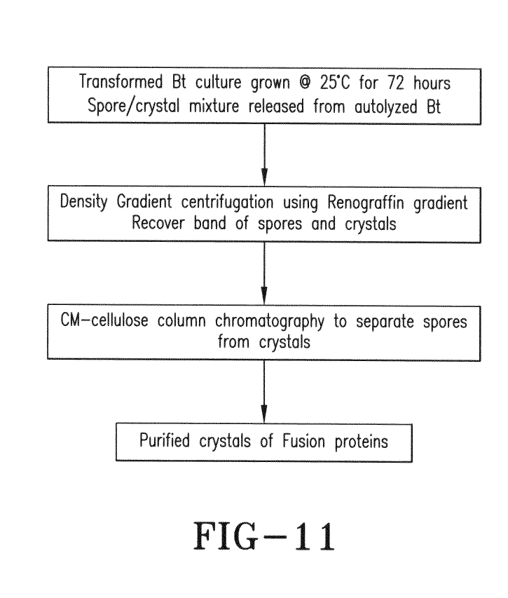

In alternative embodiments, heterologous proteins may be chemically crosslinked to a Cry crystal. In the example that follows, crystals of Cry1Ab were crosslinked using a thiol-based crosslinker: bis-maleimidoethane (BMOE) to two different mutants of the tuberculosis antigen ESAT6.
1. ESAT6-S16C
2. ESAT6-S16C bearing a T-cell helper peptide (source=vesicular stomatitis virus)
   Purified proteins of mutants 1 or 2 were produced using Ni-affinity purification method and confirmed on an SDS-PAGE gel (FIG. 13).
   Heterologous ESAT6 antigens were crosslinked to Cry1Ab crystals using the following method.
1. Crystals of Cry1Ab were produced in Bacillus thuringiensis by growing the cells in modified Schaefers sporulation medium (SSM) to autolyse the bacterium.
2. Crystals were harvested by centrifugation at 7000 rpm for 5 min and the pellet was resuspended in sterilized water.
3. Continuous density gradient medium of a contrast enhancing reagent; iodixanol was generated in water using a gradient maker.
4. The harvested pellet was centrifuged in the gradient at 5000 rpm for 70 min in Beckman L7 ultracentrifuge using a swinging bucket rotor.
5. Band containing crystals (examined using phase contrast microscopy) was extracted from the gradient and washed 7-10× with sterilized water to purify the crystals off the debris and any iodixanol. Crystals were then washed 3× and resuspended in phosphate buffered saline (PBS) pH 7.0.
6. Purified crystals were reduced using 1 mM tris(2-carboxyethyl)phosphine (TCEP) at 4° C. for 1 hr and washed off excess TCEP using PBS pH 7.0 (repeat washes for 7-10×).
7. The reduced crystals were treated with 2 fold molar excess of BMOE crosslinker and incubated at 4° C. for 3 hours. At the end of 3 hours the reaction was quenched using 0.2M DTT and crystals were washed with 5 ml PBS pH 7.0 for 10×.
8. Washed crystals were then mixed with mutant #1 or mutant #2 of ESAT6 (see above for description of mutants) in PBS and incubated @ 4° C. overnight for reacting with the crosslinked crystals.
9. Crosslinked crystals bearing ESAT6 mutants were washed with 10 ml PBS (pH 8.0) for 10×.
10. Crosslinked crystals (50 ul) was solubilized using 50 mM Na₂CO₃ (pH 10.5) in presence of 2% β-mercaptoethanol and blotted against standards of ESAT6.
11. Dot blots using anti-ESAT6 antibody were performed to quantitate the amount of crosslinking (FIG. 14).

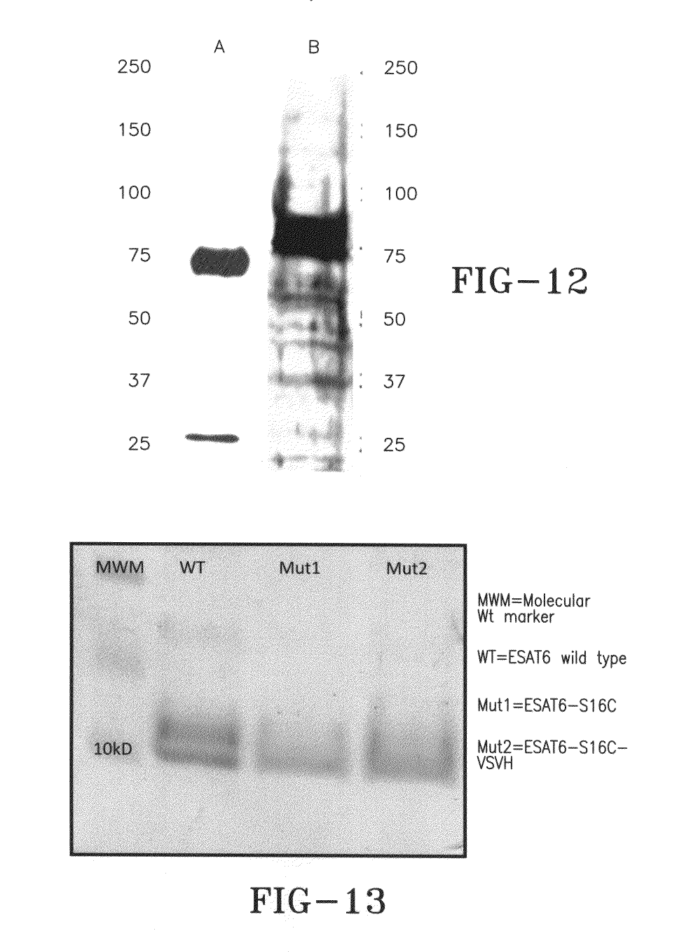

An alternative method for crosslinking an ESAT6 antigen to a Cry crytal involves the following steps:
1. Incubate pure ESAT6 protein with SIA in 1:10 molar ratio in PBS @ RT
2. Purify the ESAT6 protein off free label using PD10 (Sephadex G25) column
3. Incubate crystals of Cry1Ab with 1 mM DTT for 30 min
4. Centrifuge @ 21000 g for 10 min @ 4° C.
5. Wash with 1×PBS. Repeat centrifugation @ 21000 g. Repeat step 5 for 3×.
6. Mix the crystals of Cry1Ab with SIA crosslinked ESAT6 in molar ratio of 1:10
7. Incubate @ RT for 18-20 hours
8. Centrifuge the crystals off free ESAT protein @ 21000 g for 10 min @ 4° C.
9. Wash the crystals with 1×PBS (pH 7.5) and centrifuge @ 21000 g for 10 min @ 4° C.
10. Repeat step 6 for 3×
11. Test for crosslinking using Dot blot using anti ESAT6 primary antibody @ 1:10000 dilution
12. Used controls to measure crosslinking specificity:
   ESAT6 mixed with Cry1Ab without SIA treated same way as sample
   crystals of Cry1Ab alone (negative control)
   ESAT6 protein alone (positive control)
      In order to analyze whether the epitope was correctly crosslinked to Cry1Ab, a dot blot was performed. As demonstrated in FIG. 15 (dot blot of various ESAT6/Cry proteins), the dot blot confirmed that ESAT6 may be successfully crosslinked to a Cry protein, for example, Cry1Ab: Box 1=ESAT6 from M. marinum (fusion ESAT-1Ab expression), Box 2=Buffer control (Phosphate buffer used for crosslinking), Box 3= ESAT6 from M. tuberculosis (crosslinked to Cry1Ab crystals), Box 4=ESAT6 protein from M. tuberculosis (control), Box 5=ESAT6 protein mixed with crystals of 1Ab (not crosslinked control), Box 6=crystals of Cry1Ab (crystal control)

(iii) LcrV/Cry Fusion Protein.

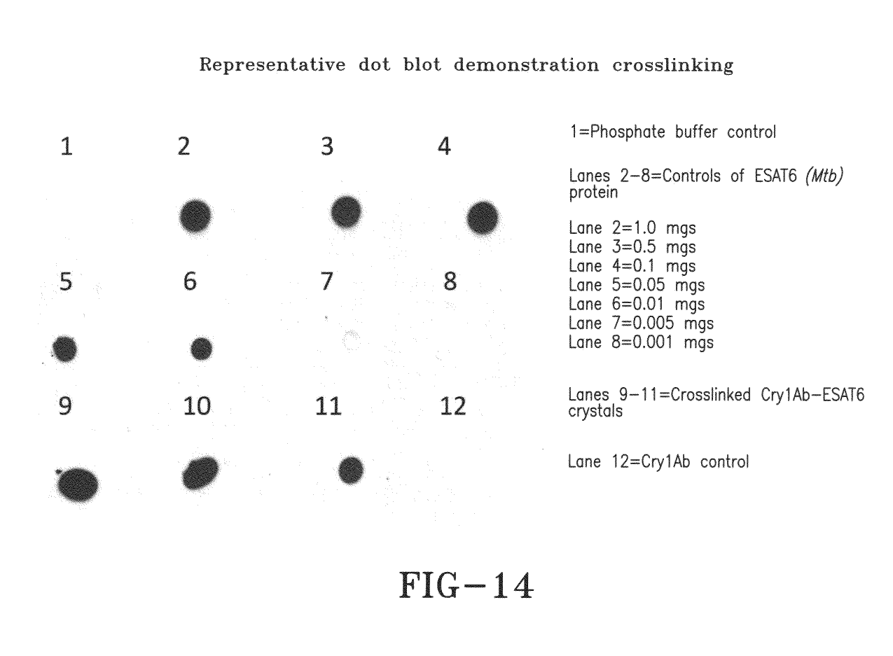

An Cry fusion with an LcrV antigen protein was constructed as described above in the pHT315 shuttle vector. As previously mentioned, LcrV is the V antigen of the pathogen, Yersina pestis. FIG. 16 shows a Western Blot using 1:10,000 dilution of anti-LcrV antibody on LcrV-Cry1Ab fusion crystals solubilized in 50 mM Na₂CO₃ pH10.5 for 1 hr (lane 2) and 2 hrs (lane 4). Controls on lane 1 and 3 include Crystals of Cry1Ab solubilized for 1 hr and 2 hrs respectively.

(iv) Antibody Response to Cry Fusion Crystals

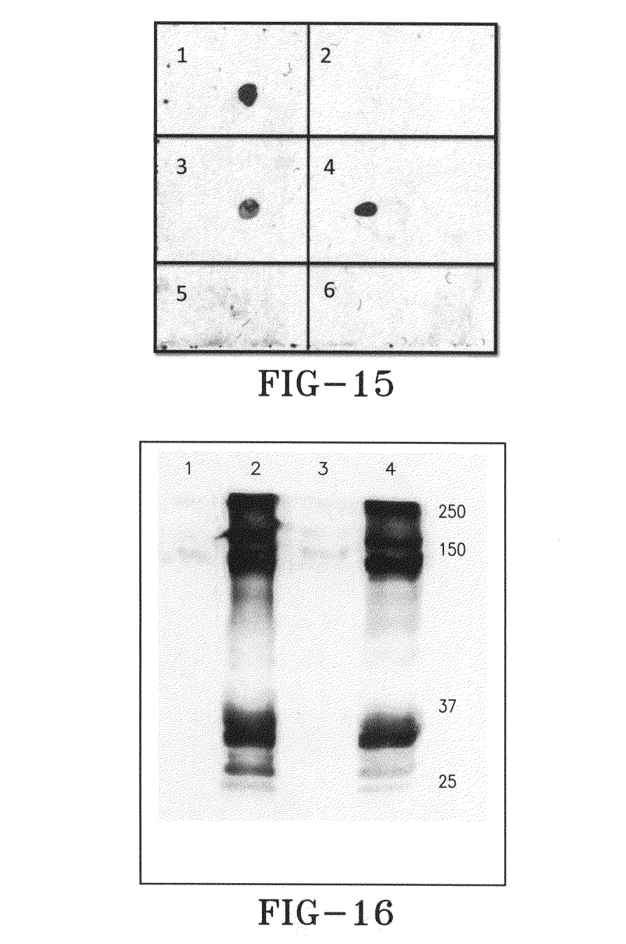

To compare the immunogenicity of the Cry-ESAT6 fusion crystals to that of the ESAT6 protein from M. marinum alone, initial antibody titre was measured in BALB/c mice. The mice were injected with equivalent doses of ESAT6 and the crystals on a fixed immunization schedule. FIG. 17 is graph comparing antibody responses in Balb/c mice toward ESAT6-TTP2/MVFP (set forth in chart as “ESAT6”) and ESAT6-Cry1Ab crystals (set forth in chart as “ESAT6-Cry”). Briefly, Mice were immunized at 0, 2, 4 weeks with 10 μg/mouse of ESAT6-Cry crystals or 50 μg/mouse of purified recombinant ESAT6-TTP2/MVFP (a known immunogen). Colorimetric ELISA assays were developed with serum diluted at 1:250 titer. The antibody response towards crystals was found to be equivalent to that of the soluble protein that was supplemented with an immune recognition helper peptide TTP2 indicating the potential ability of the crystals to act as an immunomodulator. The fusion crystals of ESAT6 reach the high titer in a much longer time than the soluble protein itself suggesting that the antigen maybe initially excluded from presentation to the immune system due to its buried location and as the proteases open up the crystal packing, there is a higher amount of antigen presented to immune system.

(v) Edible Therapeutic Agents

Japanese natto, That/Indian kinema, and West African dawadawa are foods produced by Bacillus fermentation of either soy bean or African locust beans. By using Bacillus strains engineered to make 0.3 μm antigen-crystal protein inclusions in the fermentation process, embodiments can provide cheap and edible vaccines suitable combating infectious diseases in the developing world.

Production. As previously demonstrated, the gene for the highly immunogenic antigens, such as ESAT6 from Mycobacterium tuberculosis, may be fused to a cry gene enabling the production of Cry-antigen fusion protein crystals. Following a standard recipe for Japanese natto, 100 g of soybeans may be soaked overnight and cooked to kill any exogenous bacteria. The resulting soybeans may be inoculated with a 5 mL culture of Cry-antigen fusion crystal forming Bacillus and then allowed to ferment at 37° C. for 24 to 48 hrs to yield the desired natto TB-vaccine.

Testing the therapeutic efficacy of Cry-antigen food supplement vaccines. Desired antigens may be delivered to mice using an oral immunization route. For example, the fermented product (e.g., Natto) generated using Cry-antigen crystal-producing Bacillus may be fed (at a prime dose of 10 μg/meal) to a group of 5 mice. Control mice may be fed natto generated from either Cry crystal-producing Bacillus (no antigen), or Cry deficient Bacillus (no crystals). Repeat booster doses will be provided every 3 weeks for up to 5 times. The immune response of the vaccinated mice and two control groups may be compared at specific time points using ELISA kits designed to measure the levels of specific cytokines (e.g. CD4 and TNFα) typically associated with antigen challenge.

After 4-6 weeks of immunization, the mice may be challenged with a low dose aerosol (50-100 CFU) of the virulent pathogen. A count of the viable bacilli remaining after 30 days of challenge will determine the effectiveness of protection. The approach would be to homogenize the organs (lungs and spleen) at 30 days post challenge and plate serial 10 fold dilutions on 7H11 agar plates to calculate the residual CFU.

In addition, to evaluate cytotoxic T lymphocyte responses, we will use interferon gamma (IFN-γ) and/or interleukin 2 (IL-2) enzyme-linked immunosorbent spot assays from blood samples collected 30 days after challenge. To analyze IFN-γ responses, ELISPOT assays using anti-IFN-γ antibody coated plates will be performed. Mouse splenocytes or lymphocytes from immunized mice will be incubated with ESAT6 antigen at 37° C. (in CO incubator) for 24-48 hrs. After washing off cell debris, plates will be incubated with biotinylated anti-IFN-γ-primary antibody for 2 hours, followed by streptavidin-HRP conjugate for 2 hours and colorimetric substrate. The color developed will be read using an immunospot analyzer.

(c) Agent for Delivery and Transport of Functional Species

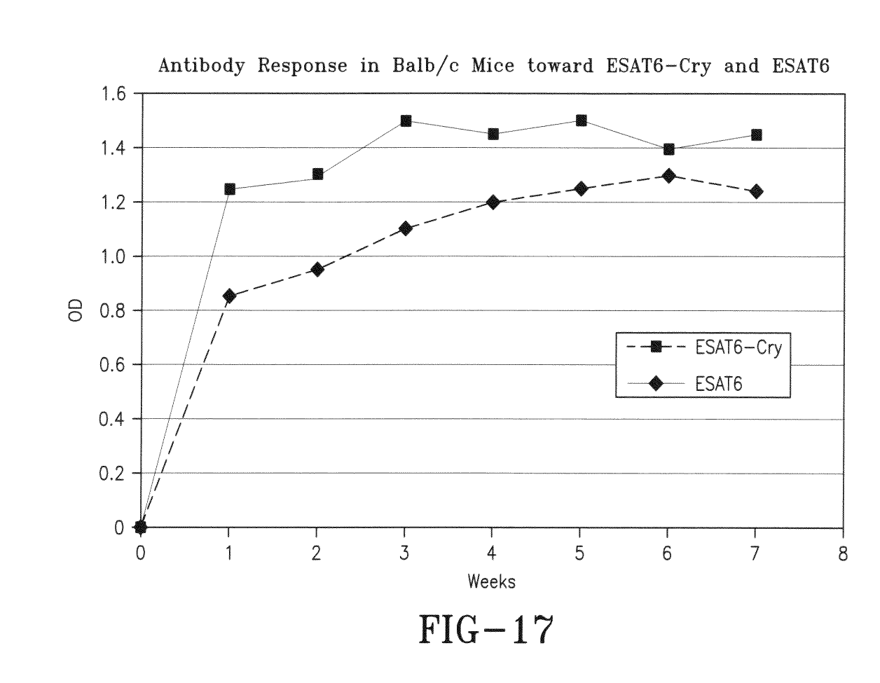

Exemplary embodiments include novel platforms for oral delivery of a functional species (e.g., an enzyme or protein therapeutic) based on regularly shaped micrometer-sized protein crystals produced within the bacterium Bacillus thuringiensis. In an exemplary embodiment, these biological protein crystals comprise Cry proteins. Overexpression in Bacillus thuringiensis of Cry proteins fused to reporter proteins such as Cry-GFP or Cry-luciferase results in the formation of protein crystals. As illustrated in FIG. 18, Cry fusion protein crystals serve as a novel platform to encapsulate target proteins within a protective crystalline framework. In this regard, two important features of these biologically-generated Cry protein crystals are (1) their relatively uniform size, and (2) their stability under standard physiological conditions.

In various embodiments, Cry proteins crystals may facilitate the delivery of various Reactive Oxygen Species (ROS)-degrading enzymes (e.g., superoxide dismutase, glutathione oxidase, catalase, etc.). Embodiments include a novel oral therapy to suppress damage from ischemia reperfusion injuries. Various compositions may also serve as an oral supplement to delay cognitive decline or extend lifespan. In other embodiments, Cry proteins crystals may facilitate the delivery of a nerve gas degradation enzyme (Transmembrane Biosciences). In another embodiment, the Cry protein crystals may facilitate the delivery of a cocaine degrading enzyme. Although oral administration is preferred, the generated Cry crystals comprising fusion polypeptides possessing functional species may be administered by other routes discussed above.

The application of enzymes as drugs is one of the newer frontiers in pharmaceutical science. One limitation of most of the enzyme replacement therapies on the market, however, is that they require intraperitoneal injection. Moreover, the lifetime of the enzyme in the vascular system is not particularly high. These features may result in extremely high costs to administer enzyme therapies ($550,000 per year for Gaucher disease patient, for life). Embodiments overcome these limitations with biological protein crystals that serve as a general platform for oral delivery of enzyme therapeutics. In various embodiments, a target enzyme therapeutic will be produced as a fusion protein to a crystal-forming Cry protein that naturally self-assembles into crystals within the bacterium Bacillus thuringiensis. Advantageously, embodiments include a general enzyme delivery platform that: (1) can facilitate oral or nasal administration, (2) is cheap, (3) is pure and uniform in size, and (4) protects the target enzyme from proteolytic degradation.

At least one embodiment comprises a Cry crystal fusion protein to treat pathological conditions induced by reactive oxygen species (ROS) (e.g., overproduction of superoxide and hydrogen peroxide). In humans and most other organisms, there are specific enzymes to remove and degrade ROS. Degradation of superoxide, the most reactive ROS species, is mediated by superoxide dismutases (SOD). These enzymes convert the radical superoxide into the milder oxidants, dioxygen and hydrogen peroxide. Hydrogen peroxide, in turn, is degraded by catalases via disproportionation or by glutathione peroxidases (GPx) that use the oxidizing equivalents of hydrogen peroxide to oxidize glutathione (GSH). Notably, exogenous treatment with these enzymes has been shown to have numerous benefits including increased life span, delay of cognitive decline due to aging, and protection against oxidative cellular damage. Despite its promise, there is no enzyme-based ROS therapy currently in the market. Accordingly, embodiments comprise Cry crystal-enzyme fusion proteins such as Cry-SOD and Cry-GPx crystals, for use as oral therapeutics and supplements.

Embodiments comprise a novel platform as a vehicle to deliver enzyme therapeutics to the vascular system. This platform is based on micrometer-sized protein crystals that are naturally produced within the cells of the Gram-positive bacterium, B. thuringiensis. These crystals are made up of a specific class of crystal-forming proteins called Cry proteins. Depending on the crystal size, each crystal contains 150-500 Cry protein molecules.

Various embodiments incorporate a target heterologous protein or enzyme into the Cry crystal platform by fusing the gene of the target protein or enzyme to either the 5′- or 3′-end of a cry gene. In an exemplary embodiment, the fusion cassette may be cloned into an expression vector, such as the pHT315 E. coli-B. thuringiensis shuttle vector. This vector can be transfected into a bacterium and used to produce a Cry-enzyme fusion crystals within the live bacterium (e.g., B. thuringiensis). Notably, since B. thuringiensis autolyses following sporulation and the crystals have a distinct density, they can be easily purified by centrifugation. The direct synthesis of these crystals in bacteria combined with their ease of purification makes the production of these Cry crystal based therapeutics very economical.

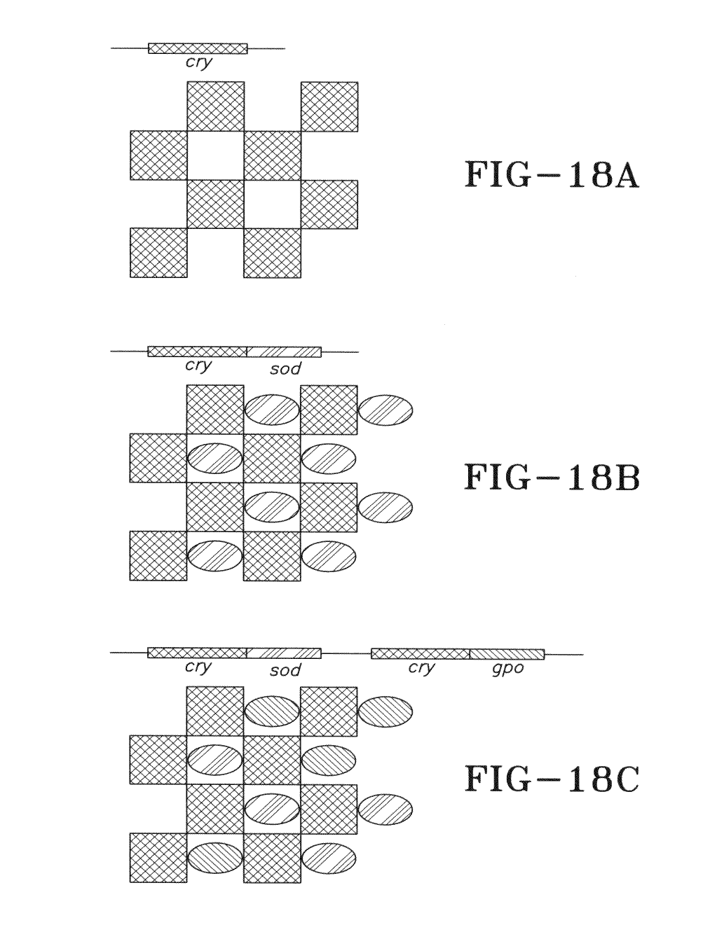

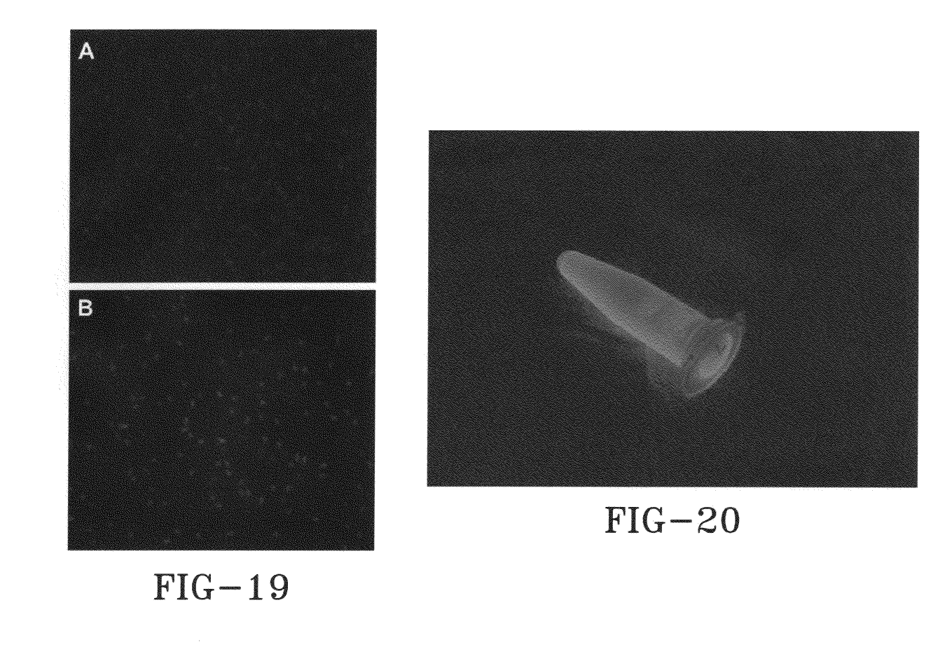

As demonstrated in FIG. 19, generated Cry-GFP and Cry-mCherry crystals are fluorescent, demonstrating the presence of the additional protein component does not hinder crystal formation and the GFP domain in the crystal is properly folded (FIG. 19). Additionally, active enzymes can also be incorporated in a similar fashion. Using the pHT315 expression vector (FIG. 3A), the gene encoding the enzyme luciferase (SEQ ID NO: 31) was fused to a gene encoding Cry1Ab (SEQ ID NO: 3). This vector was used to generate Cry-luciferase crystals as described above. Treatment of these fusion crystals with luciferin resulted in the chemiluminescence expected for active luciferase (FIG. 20).

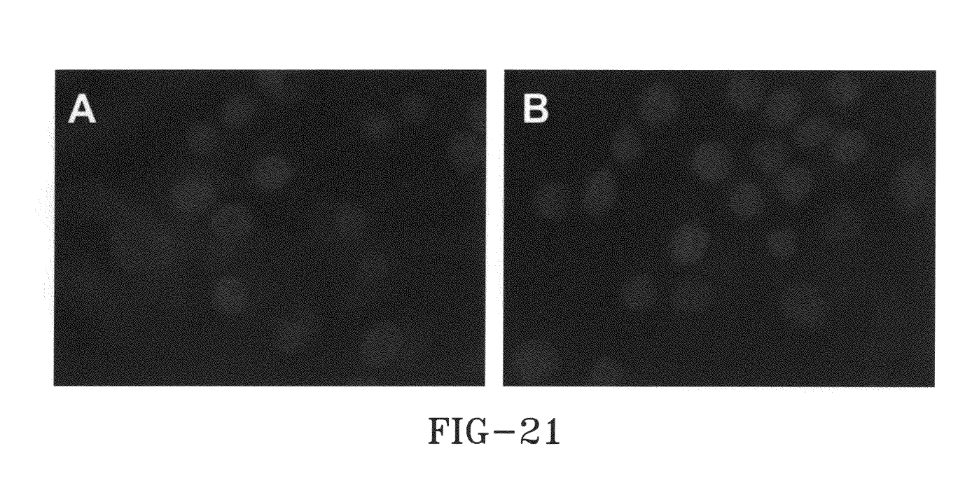

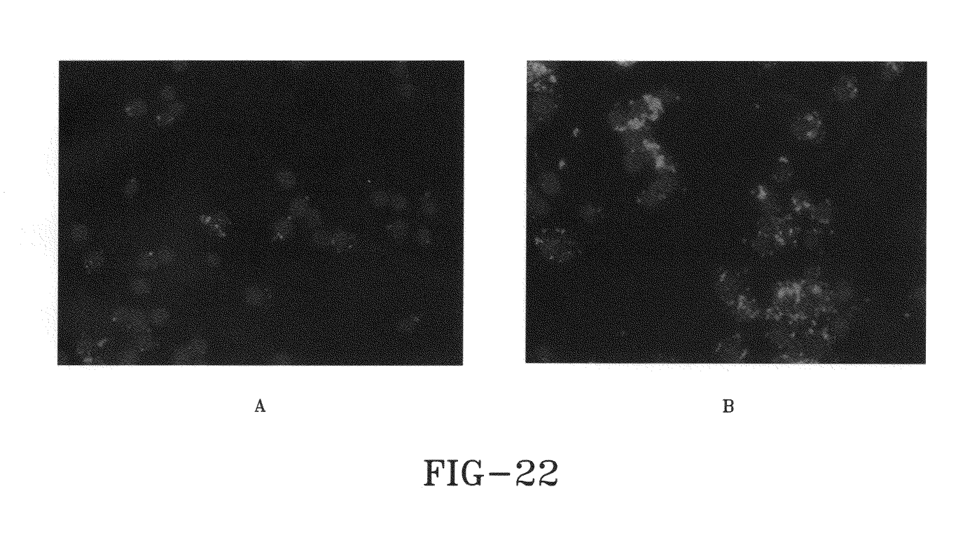

While Cry crystals are inert to most cells, they may be taken up by macrophages. Various embodiments apply a PEGylation approach to hinder phagocytosis of Cry crystals. Towards this end, PEGylated Cry-GFP crystals have been prepared and then used to demonstrate that PEGylation reduces phagocytosis as expected (FIG. 21). In exemplary embodiments, PEG sizes should be chosen that minimizes macrophage phagocytosis, and yet are optimal for catalytic chemistry. In alternative embodiments, it may be advantageous to allow macrophages to take up cry crystal fusion proteins, for example, to deliver a therapeutic enzyme. Accordingly, FIG. 22 demonstrates the short-term uptake of Cry-GFP crystals by macrophages. The fluorescent image shows the uptake of crystals by macrophages at 15 minutes (A) and 4 hrs (B). These images are of Cry3A-GFP crystals. The blue is the nucleus. The green dots are the fluorescent crystals being taken up by the macrophage in these images.

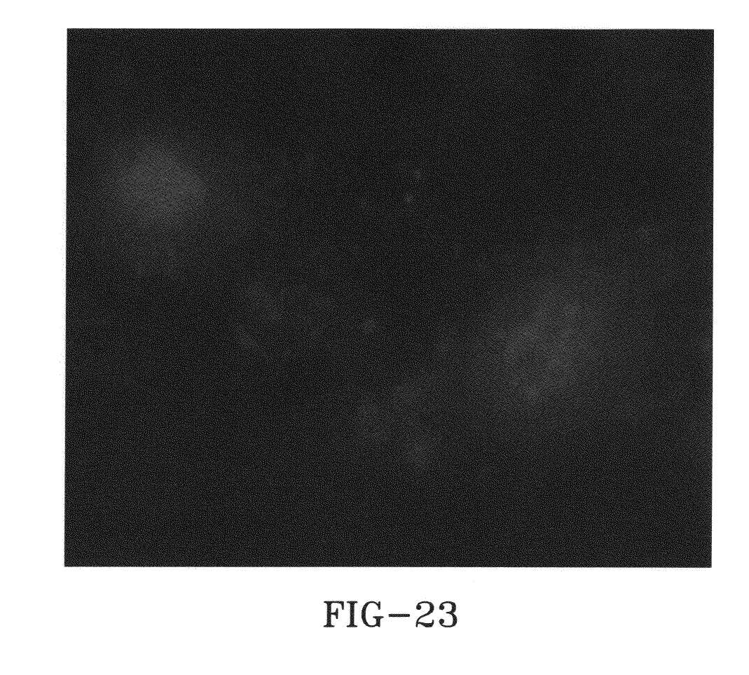

Besides macrophages, other cell types may readily take up Cry crystal fusion proteins. To demonstrate this capacity, NIH3T3 fibroblasts were incubated with 15 uL 0.6 ug/mL Cry3Aa-mCherry crystals in 200 uL DMEM complete media for 1.5 hrs at 37° C./5% CO₂. At the end of the incubation, cells were washed with 1 mL 1×PBS three times to get rid of any free Cry3Aa-mCherry crystals. 200 uL of DMEM complete media were then added to the washed cells for a further 1.5 hrs incubation before fixation with paraformaldehyde. As can be seen in fluorescence micrograph of FIG. 23, the fibroblasts clearly contain the Cry3Aa-mCherry crystals (see red spots surrounding the DAPI stained nuclei).

Exemplary embodiments comprising Cry crystal fusion proteins, like Cry crystals themselves should have tremendous stability. Specific embodiments comprise proteins and enzymes fused to Cry (e.g., Cry1Ab) crystals. Many Cry crystals require high pH to solubilize the crystal, even in the presence of proteases. Given the acidic nature of the human gastrointestinal tract, it is expected that the embodied crystal will remain intact. Accordingly, the Cry crystal will be able protect its enzyme cargo from proteolytic degradation. Alternative embodiments use crosslinking and/or other surface modifications to link Cry crystals to relevant enzymes.

In addition to stability, another consideration for oral delivery is the mechanism of uptake. In this regard, one important advantage of Cry crystals is they are composed of Cry proteins, a protein which insert into the membranes of insect midgut cells. Because of this role, embodied Cry crystal fusion proteins may have beneficial properties to aid in crystal transport across the intestinal wall. In alternative embodiments, a variety of transport enhancers such as chitosan derivatives that have been shown to assist in paracellular uptake of proteins may be attached.

Embodiments include Cry-SOD and Cry-SOD/GPx crystals as enzyme therapeutics. In various embodiments, the gene corresponding to the E. coli Cu—Zn SOD (SEQ ID NO: 30) may be fused to a cry gene (e.g., SEQ ID NO: 3) in the B. thuringiensis expression vector, pHT315. The E. coli Cu—Zn SOD is ideal because it is relatively small (17 kDa), highly active, and has been confirmed to be monomeric based on its crystal structure.

Since degradation of superoxide by SOD enzymes produces hydrogen peroxide, a milder but still potent ROS, at least one embodiment comprises a Cry-SOD/GPx cocrystal generated by coexpression of the Cry-SOD and Cry-GPx genes. The glutathione peroxidase will take any hydrogen peroxide generated by SOD and use it to promote the oxidation of glutathione, an abundant blood metabolite. The choice of GPx over catalase stems from its smaller size and the fact that it does not have a requirement for manganese—a mutagen due to its effect on the fidelity of DNA polymerases. Various embodiments use the GPx from Bacillus thuringiensis as it is not a selenoprotein based on sequence alignments and has the best chance of being produced in its active form as it is from the organism the crystals are produced in.

Embodied crystals may be intraperitonially injected and/or orally administered Cry-SOD/GPx crystals to protect against myocardial reperfusion injury, aging, metabolic syndrome, and other diseases where ROS are implicated.

Various embodiments utilize a Cry crystal platform in a unique approach for oral delivery of enzyme therapeutics. Prior to the innovations described, protein crystal technology required multiple steps to produce the target protein. With past methods and systems, the protein needed to be purified, then the crystallization conditions needed to be identified before the protein could be crystallized. Finally, many past technologies required the proteins to be crosslinked to maintain stability in the vasculature system. Advantageously, in various embodiments described herein, all of these steps can now be performed by the bacterial host, with the sole step being purification of the crystals by sucrose gradient centrifugation.

(d) Cry Crystals as a Blood Substitute

Research concerning blood substitutes is significant for many reasons and offers many benefits over human blood transfusions, particularly in certain high trauma situations. Blood substitutes reach full oxygen transport capacity immediately while transfused blood can take 24 hours to reach its full capacity. Because oxygen-carrying blood substitutes do not have any blood antigens, they can be used for all blood types without causing a negative immunologic response for rapid treatment of trauma victims. Additionally, a disease-free source of oxygen therapeutics would greatly benefit regions of the world where HIV/AIDS affects a large portion of the population and the blood supply is relatively unsafe.

Myoglobin (Mb) is a single-chain globular protein of 153 amino acid residues and one heme molecule. This oxygen-binding protein facilitates oxygen diffusion in mammalian muscle tissue. Myoglobin's structure is very similar to that of both the alpha and beta hemoglobin subunits. However unlike hemoglobin, oxygen binding of myoglobin is relatively unaffected by the pressure of oxygen in surrounding tissues. It binds oxygen with high affinity but cannot change its affinity like the multimeric hemoglobin can. Therefore myoglobin has a hyperbolic binding curve for oxygen and is normally better suited for oxygen storage than for oxygen transport.

A successful Mb-based blood substitute will need selective variation in its affinity for oxygen, imitating the versatility in O₂ affinity that hemoglobin achieves through changing its conformation. The necessary lower oxygen affinity can be accomplished in myoglobin either by increasing steric hindrance of the bound oxygen or by weakening the hydrogen bonding in the polar iron-oxygen complex of myoglobin. Therefore embodiments incorporate double mutants of myoglobin into the blood substitute that will be able to increase and decrease oxygen affinity. Next, the myoglobin mutant may be fused to a crystal-forming Cry protein which is produced by Bacillus thuringeniesis bacterium.

In exemplary embodiments, a Cry-myoglobin fusion protein forms crystals exhibiting the oxygen binding characteristics of the myoglobin mutants. In the embodiments, the resulting crystal encapsulated myoglobin mutants may have properties suitable for oxygen transport, stability for long-term use while patient blood levels are recovering, and low toxicity to the human body. Notably, Cry crystals useful for the embodiments are non-toxic to humans and are approximately 1 micrometer in size—smaller than the diameters of veins and arteries of the human vascular system.

Alternative embodiments may utilize other agents as a blood substitute. For example, various embodiments may employ Cry-protein operably linked with perfluorocarbons (PFCs) and hemoglobin-based oxygen carriers (HBOCs) to form crystals exhibiting appropriate oxygen binding characteristics. PFCs are chemical compounds that can transport and release oxygen; PFC particles are significantly smaller than human red blood cells (RBCs), allowing them to reach capillaries in damaged tissue RBCs cannot reach.

(e) Cry Crystals for Stabilizing Industrial Enzymes

The ability of Cry crystals to prepare encapsulated proteins such as luciferase also speaks to its ability to encapsulate enzymes in general. Given its ease of purification, another application could be for the facile encapsulation of enzymes for chemical synthesis of molecules or for preparing stabilized variants, such as proteases and other enzymes in commercial detergents (Tide, Cheer, etc).

(f) Protein Delivery for Cell Reprogramming

Various embodiments comprise fusion protein crystals that may be used as a platform to deliver proteins intended for cellular reprogramming into cells.

In an exemplary method, the cell line of interest (e.g. macrophages) may be seeded at 5×1⁰⁴ cells/well on an 8-well chamber slide and incubated overnight at 3⁷° C. in 5% C_{O2}. Cells may then be washed with 1× phosphate-buffered saline (PBS) to remove non-adherent cells, followed by incubation with 120 μg/mL of Cry3Aa-target fusion protein (e.g. Cry3Aa—AMP-activated protein kinase) crystals in 200 uL Dulbecco's modified Eagle's medium (DMEM) supplemented with 10% fetal bovine serum and pen/strep for 24-72 hours at 3⁷° C. in 5% C_{O2}. At the end of the incubation period, the cells may be washed three times with PBS to remove any free Cry3Aa-target fusion protein crystals, and then characterized for phenotypic properties.

The ability to deliver functional peptides offers many therapeutic avenues. For example, others have shown that that exogenous expression of the germline-specific transcription factor Oct4 is sufficient to generate pluripotent stem cells from adult mouse NSCs. See Kim, et al. Cell 136, 411-419, Feb. 6, 200; Zhou et al. Generation of Induced Pluripotent Stem Cells Using Recombinant Proteins. Cell Stem Cell 4, May 8, 2009. However, past approaches have been limited by the technological obstacles to gene and protein delivery.

Cry crystals provide an efficient means for delivering reprogramming polypeptides. In one prophetic example, the limitations may be overcome by delivering the reprogramming proteins (e.g., Oct4 protein) in the form of a Cry fusion Crystal. For the pHT315 vector could be used to create a Cry3Aa-Oct4 fusion crystal expression vector. Similar to the expression vector shown in FIG. 3B, a cry3Aa coding sequence (SEQ ID NO: 7) from Bacillus thuringienis may be fused upstream of the Oct4 gene (SEQ ID NO: 33) followed by a stop codon. The protein can then be contacted to neuronal stem cells to facilitate reprogramming.

Functional inactivation of the tumor suppressor protein p16^{INK4a} has been demonstrated to be an important aspect in the transformation of pancreatic ductal cells as well as hematopoietic cells. However, to date targeting of associated malignancies has been hindered by technological obstacles to delivering nucleic acids and peptides in the neoplastic cells. Accordingly, various embodiments comprise a method and a composition for protein delivery whereby a fusion polypeptide comprising a Cry protein fused to a tumor suppressor protein, such as p16^{INK4a} may be efficiently introduced into diseased cells and tissue. In alternative embodiments, the protein crystal may be crosslinked or bound to the tumor suppressor protein, such as p16^{INK4a}. In this example, the cry3Aa coding sequence (SEQ ID NO: 7) from Bacillus thuringienis may be fused upstream of the p16^{INK4a} coding sequence (SEQ ID NO: 34) followed by a stop codon. The p16^{INK4a} protein can then be delivered to malignant cells in order to inhibit metastasis.

### OTHER EMBODIMENTS

It is to be understood that while embodiments have been described in conjunction with the detailed description thereof, the foregoing description is intended to illustrate and not limit the scope of the invention. Other aspects, advantages, and modifications are within the scope of the following claims.

1. A cultured cell, comprising:
a protein crystal formed by a plurality of a fusion polypeptide, the fusion polypeptide comprising a Cry protein, or a crystal-forming fragment thereof, fused to a heterologous polypeptide.

2. The cultured cell of claim 1, wherein:
the cell is a bacterial cell.

3. The cultured cell of claim 1, wherein:
the cell is a plant cell.

4. The cultured cell of claim 2, wherein:
the heterologous polypeptide is an immunogenic antigen.

5. The cultured cell of claim 2, wherein:
the heterologous polypeptide is an imageable agent.

6. The cultured cell of claim 5, wherein:
the imageable agent is a fluorescent protein.

7. The cultured cell of claim 2, wherein:
the heterologous polypeptide is a blood substitute.

8. The cultured cell of claim 2, wherein:
the heterologous polypeptide is a therapeutic enzyme.

9. The cultured cell of claim 2, wherein:
the Cry protein is selected from the group consisting of Cry1Aa, Cry1Ab Cry2Aa, Cry3Aa, Cry4Aa, Cry4Ba, Cry11Aa, Cry11Ba, and Cry19Aa, their homologs, or a crystal forming fragment thereof.

10. The cultured cell of claim 2, wherein:
the antigen is selected from the group consisting of fbpA, fbpB, fbpC, ESAT6, erp (pirG), Rv1477, MPT53, OmpAtb, IiA, p60, MPT53, OspA.

11. A protein crystal isolated from a bacterium, comprising:
a fusion polypeptide, the fusion polypeptide comprising a Cry protein fused to a heterologous polypeptide.

12. The protein crystal of claim 11, wherein:
the heterologous polypeptide is an immunogenic antigen.

13. The protein crystal of claim 11, wherein:
the heterologous polypeptide is an imageable agent.

14. The protein crystal of claim 13, wherein:
the imageable agent is a fluorescent protein.

15. The protein crystal of claim 11, wherein:
the heterologous polypeptide is a blood substitute.

16. The protein crystal of claim 11, wherein:
the heterologous polypeptide is a therapeutic protein or enzyme.

17. A composition, comprising:
a Cry protein crystal chemically crosslinked to a heterologous polypeptide.

18. A nucleic acid comprising a nucleotide sequence encoding a fusion polypeptide capable of forming crystals in vivo in a cell, the fusion polypeptide comprising a Cry protein fused to a heterologous polypeptide.

19. An expression vector comprising the nucleic acid of claim 17.

20. A bacterial endospore comprising a nucleic acid comprising a nucleotide sequence that encodes a fusion polypeptide, the fusion polypeptide comprising a Cry protein and a heterologous polypeptide.

21. A fusion polypeptide comprising a Cry protein and an immunogenic antigen.

22. A pharmaceutical composition comprising the protein crystal of claim 11 and a pharmaceutically acceptable excipient, carrier, diluent, or vehicle.

23. A pharmaceutical composition comprising the protein crystal of claim 17 and a pharmaceutically acceptable excipient, carrier, diluent, or vehicle.

24. A method of isolating a recombinant protein crystal from a bacterium, the method comprising:
transforming the bacterium with a nucleic acid expression vector encoding a Cry protein fused to a heterologous polypeptide;
growing the bacterium in culture until a spore/crystal mixture is released from the bacterium upon autolysis;
centrifuging the spore/crystal mixture using a density gradient or affinity method; and
isolating the purified crystals of fusion proteins.

25. The method of claim 24, wherein:
the bacterium is a Bacillus thuringiensis or a Bacillus subtilis.

26. A protein crystal obtained from the method of claim 24.

27. A method of eliciting an immune response against an antigen in a subject, the method comprising administering the protein crystal of claim 11 to a subject in an amount effective to induce an immune response against the antigen in the subject.

28. The method of claim 27, wherein the administering step is performed intranasally, orally, or intraperitoneally.
